(19) United States
(12) Patent Application Publication
(10) Pub. No.: US 2024/0018426 A1
(43) Pub. Date: Jan. 18, 2024

(54) Process for manufacturing bio-based hydrocarbons

(71) Applicant: Neste Oyj, Espoo (FI)

(72) Inventors: Imane HACHEMI, Porvoo (FI); Andres MUNOZ GANDARILLAS, Porvoo (FI); Lars BÖRGER, Porvoo (FI); Pekka AALTO, Porvoo (FI); Antti OJALA, Porvoo (FI); John JAMIESON, Porvoo (FI)

(73) Assignee: Neste Oyj, Espoo (FI)

(21) Appl. No.: 18/251,449

(22) PCT Filed: Oct. 29, 2021
(86) PCT No.: PCT/FI2021/050734

§ 371 Date: May 2, 2023

(30) Foreign Application Priority Data
Nov. 6, 2020 (FI) 20206120

(51) Int. Cl. C10G 69/04; C07C 4/06; C08F 210/06; C10L 1/06; C10G 3/00

(52) U.S. Cl. CPC C10G 69/04; C07C 4/06; C08F 210/06; C10L 1/06; C10G 3/42; C10G 2400/02; C10G 2300/1014; C10G 2300/1018; C10G 2300/305; C10G 2300/301; C10L 2270/023; C10L 2200/0469

(57) ABSTRACT

The present disclosure relates to a process for manufacturing bio-based hydrocarbons, such as bio-propylene and optionally bio-gasoline, and to a bio-propylene composition, a bio-gasoline component and to a method of producing a (co)polymer composition. The process can include hydrotreating an oxygen-containing bio-based feedstock, followed by gas-liquid separation and optionally fractionation, to provide a hydrotreated bio-based hydrocarbon feed containing less than 1 wt.-% of gaseous compounds (NTP), providing a catalytic cracking feed containing the hydrotreated bio-based hydrocarbon feed; catalytically cracking the catalytic cracking feed in a catalytic cracking reactor at a temperature of at least 450° C. using a moving solid catalyst to obtain a cracking effluent; and recovering from the cracking effluent a fraction rich in bio-propylene.

### TECHNICAL FIELD

The present disclosure generally relates to catalytic cracking. The disclosure relates particularly, though not exclusively, to catalytic cracking of a hydrotreated bio-based hydrocarbon feed using a moving solid catalyst to manufacture a bio-propylene composition, and optionally a bio-gasoline component. Further, the invention relates to a bio-propylene composition and a bio-gasoline component and to a method of producing a (co)polymer composition.

### BACKGROUND

This section illustrates useful background information without admission of any technique described herein representative of the state of the art.

The global demand for propylene is huge, and expected to continue increasing due to growing demand for polypropylene. Propylene is the second largest volume chemical produced globally and is an important raw material for the production of many organic chemicals such as polypropylene, acrylonitrile, propylene oxide, oxo alcohols and a large variety of industrial products. The major production routes include well known petrochemical processes like steam cracking and refinery FCC units producing propylene as a by-product of ethylene and of liquid fuels, respectively. Also on purpose technologies like propylene dehydrogenation (PDH) are increasing in capacity, but steam cracking remains as the dominant technology for producing propylene, being the number 2 process in the chemical industry as judged by the scale. Steam cracking of petroleum-based feedstocks generates ethylene (C2=) as the main product and propylene as the desired by-product, and also waste by-products such as CH₄, CO and CO₂. Steam cracking is one of the most energy-intensive industrial processes.

Catalytic cracking processes using fluidized solid catalyst, e.g. FCC units, are well known petrochemical processes, and have been used for decades for processing fossil feeds, predominantly into liquid fuels. Initially, it was researchers of the Standard Oil of New Jersey who developed the first fluidized catalytic cracking unit, and were awarded a patent U.S. Pat. No. 2,451,804 “A Method of and Apparatus for Contacting Solids and Gases”. Based on their work, a large pilot plant was constructed in 1940, followed by a first commercial fluid catalytic cracking plant in 1942. Since then, the process designs and usable feedstocks for fluid catalytic cracking processes have evolved greatly. Examples of developed FCC designs include High Severity Fluid Catalytic Cracking (HS-FCC) and Deep Catalytic Cracking (DCC), as well as on-purpose olefins manufacturing processes, such as ExxonMobil PCCSM and KBR Superflex.

The awareness of greenhouse gas emissions and environmental concerns relating to oil drilling and petroleum refining is increasing, but at the same time there is no end in sight to the increase of fuels and chemicals demand, so alternative feed sources must be investigated diligently. The abundance and sustainability of biomass makes it an attractive option to supplement the future demand for petroleum, but it involves the challenge of having high oxygen content. Various thermal and thermocatalytic schemes have been proposed for production of liquid hydrocarbon fuels from bio-based materials. Even direct processing of solid biomass or other oxygenated carbonaceous feedstocks by catalytic cracking using fluidized solid catalyst has been investigated in an effort to directly deoxygenate the biomass and produce transportation fuels and other hydrocarbons, e.g. in U.S. Pat. Nos. 5,792,340A and 5,961,786A.

Other alternatives for hydrocarbon production from bio-based materials include converting solid biomass first into a thermally or thermocatalytically produced oxygenates-containing liquid, and then feeding the liquid into a circulating fluid bed reactor using e.g. a FCC catalyst as the solid circulating media (Adjaye et al., Production of Hydrocarbons by Catalytic Upgrading of a Fast Pyrolysis Bio-oil, Fuel Processing Technology 45 (1995), 185-192).

Although some hydrocarbon products have been obtained using these approaches, the yields of the desired products have been low, yields of char/coke and by-product gases high, and often accompanied by other issues relating e.g. to reactor fouling and plugging, and to catalyst performance. Additionally, the produced liquid product would require further upgrading and treatment to enable direct immediate use in place of fossil liquid fuels.

As a means to overcome the technical and economic limitations associated with full stand-alone biomass upgrading to transportation fuels, researchers (e.g. de Miguel Mercader, Pyrolysis Oil Upgrading for Co-Processing in Standard Refinery Units, Ph.D Thesis, University of Twente, 2010) have been looking at partial upgrading of the oxygenated biomass to reduce oxygen, followed by co-processing of the intermediate biomass product with petroleum feedstocks in existing petroleum refinery units. These initiatives are focused on hydrodeoxygenation of the biomass-derived liquid prior to co-processing with petroleum, in order to avoid rapid FCC catalyst deactivation and reactor fouling, and to reduce excessive coke and gas formation.

Despite of the fact that inexpensive biomass sources are abundantly available, the requirement for several multi-step processings of the biomass for converting it first to intermediates that are eventually capable of being processed into valuable fuel and chemical end-products, makes bio-based raw materials less attractive from the economical point of view. Furthermore, while the approaches involving co-processing of bio-based materials with petroleum feedstocks increase sustainability of the products to certain degree, 100% bio-based fuels and chemicals cannot be achieved. Thus, there is a continuous need for more efficient processing of the bio-based raw materials into high quality chemicals and fuels, wasting less of the valuable raw material, and providing higher yields of the desired high quality products.

### SUMMARY

The present invention was made in view of the above-mentioned problems and it is an object of the present invention to provide an improved process for producing bio-based hydrocarbons, as well as an improved bio-propylene composition and bio-gasoline component.

In brief, the present disclosure relates to one or more of the following items:
1. A process for manufacturing a bio-propylene composition, the process comprising the following steps (A) to (D):
(A) hydrotreating an oxygen-containing bio-based feedstock to obtain a hydrotreatment effluent comprising oxygen-depleted hydrocarbons, and subjecting the hydrotreatment effluent to a gas-liquid separation, to provide a hydrotreated bio-based hydrocarbon feed containing less than 1 wt.-%, preferably less than 0.8 wt.-%, more preferably less than 0.5 wt.-%, of gaseous compounds (NTP);
(B) providing a catalytic cracking feed comprising the hydrotreated bio-based hydrocarbon feed;
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in bio-propylene as the bio-propylene composition.
2. A process for manufacturing a bio-propylene composition, the process comprising the following steps (B′), (C) and (D):
(B′) providing a catalytic cracking feed comprising a hydrotreated bio-based hydrocarbon feed containing less than 1 wt.-%, preferably less than 0.8 wt.-%, more preferably less than 0.5 wt.-%, of gaseous compounds (NTP);
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in bio-propylene as the bio-propylene composition.
3. The process according to item 2, further comprising a step (A) of preparing the hydrotreated bio-based hydrocarbon feed by hydrotreating an oxygen-containing bio-based feedstock to obtain a hydrotreatment effluent comprising oxygen-depleted hydrocarbons, and subjecting the hydrotreatment effluent to a gas-liquid separation.
4. The process according to any one of the preceding items, wherein the oxygen-containing bio-based feedstock comprises one or more selected from the group consisting of vegetable oils, animal fats, microbial oils, thermally liquefied biomass and enzymatically liquefied biomass.
5. The process according to any one of the preceding items, wherein the oxygen-containing bio-based feedstock comprises one or more selected from the group consisting of vegetable oils, animal fats and microbial oils.
6. The process according to any one of the preceding items, wherein the hydrotreating in the step (A) comprises at least deoxygenation and isomerization.
7. The process according to item 6, wherein the deoxygenation comprises at least hydrodeoxygenation.
8. The process according to item 7, wherein hydrodeoxygenation and isomerization are conducted simultaneously in the same hydrotreating step, and/or separately in subsequent hydrotreating steps
9. The process according to any one of the preceding items, wherein the step (A) comprises subjecting the hydrotreatment effluent to a gas-liquid separation and further to a fractionation to provide the hydrotreated bio-based hydrocarbon feed containing less than 1 wt.-% of gaseous compounds (NTP).
10. The process according to any one of the preceding items, wherein the hydrotreated bio-based hydrocarbon feed comprises isoparaffins.
11. The process according to any one of the preceding items, wherein the hydrotreated bio-based hydrocarbon feed comprises, based on the total weight of the hydrotreated bio-based hydrocarbon feed, more than 1 wt.-% isoparaffins, preferably more than 4 wt.-%, such as more than 5 wt.-% isoparaffins.
12. The process according to any one of the preceding items, wherein the hydrotreated bio-based hydrocarbon feed comprises, based on the total weight of the hydrotreated bio-based hydrocarbon feed, more than 30 wt.-%, such as more than 40 wt.-% or more than 50 wt.-% or more than 60 wt.-%, even more preferably more than 70 wt.-%, such as 80 wt.-%, particularly more than 85 wt.-% isoparaffins.
13. The process according to any one of the preceding items, wherein the hydrotreated bio-based hydrocarbon feed comprises isoparaffins and n-paraffins and the sum of the wt.-% amounts of isoparaffins and n-paraffins in the hydrotreated bio-based hydrocarbon feed is at least 40 wt.-%, preferably more than 50 wt.-%, such as more than 60 wt.-%, more preferably more than 70 wt.-%, such as more than 80 wt.-%, particularly more than 90 wt.-% or even more than 95 wt.-%, based on the total weight of the hydrotreated bio-based hydrocarbon feed.
14. The process according to any one of the preceding items, wherein the hydrotreated bio-based hydrocarbon feed comprises less than 25 wt.-% total aromatics, preferably less than 15 wt.-%, more preferably less than 5 wt.-%, most preferably less than 1 wt.-% total aromatics, based on the total weight of the hydrotreated bio-based hydrocarbon feed.
15. The process according to any one of the preceding items, wherein the hydrotreated bio-based hydrocarbon feed comprises, based on the total weight of the hydrotreated bio-based hydrocarbon feed, less than 80 wt.-% naphthenes, preferably less than 50 wt.-%, such as less than 30 wt.-%, more preferably less than 10 wt.-%, most preferably less than 5 wt.-%, particularly less than 1 wt.-% naphthenes.
16. The process according to any one of the preceding items, wherein the hydrotreated bio-based hydrocarbon feed comprises, based on the total weight of the hydrotreated bio-based hydrocarbon feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C11.
17. The process according to any one of the preceding items, wherein the hydrotreated bio-based hydrocarbon feed comprises, based on the total weight of the hydrotreated bio-based hydrocarbon feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C14.
18. The process according to any one of the preceding items, wherein the hydrotreated bio-based hydrocarbon feed comprises, based on the total weight of the hydrotreated bio-based hydrocarbon feed:
   isoparaffins and n-paraffins and the sum of the wt.-% amounts of isoparaffins and n-paraffins in the hydrotreated bio-based hydrocarbon feed is at least more than 80 wt.-%, preferably more than 90 wt.-% or even more than 95 wt.-%;
   more than 80 wt.-%, preferably more than 90 wt.-% hydrocarbons having a carbon number of at least C11; and
   more than 4 wt.-%, such as more than 5 wt.-%, preferably more than 30 wt.-% isoparaffins.
19. The process according to any one of the preceding items, wherein the hydrotreated bio-based hydrocarbon feed comprises, based on the total weight of the hydrotreated bio-based hydrocarbon feed:
   isoparaffins and n-paraffins and the sum of the wt.-% amounts of isoparaffins and n-paraffins in the hydrotreated bio-based hydrocarbon feed is at least more than 80 wt.-%, preferably more than 90 wt.-% or even more than 95 wt.-%;
   more than 80 wt.-%, preferably more than 90 wt.-% hydrocarbons having a carbon number of at least C14; and
   more than 4 wt.-%, such as more than 5 wt.-%, preferably more than 30 wt.-% isoparaffins.
20. The process according to any one of the preceding items, wherein the hydrotreated bio-based hydrocarbon feed comprises, based on the total weight of the hydrotreated bio-based hydrocarbon feed:
   isoparaffins and n-paraffins and the sum of the wt.-% amounts of isoparaffins and n-paraffins in the hydrotreated bio-based hydrocarbon feed is at least more than 80 wt.-%, preferably more than 90 wt.-% or even more than 95 wt.-%;
   more than 80 wt.-%, preferably more than 90 wt.-%, more preferably more than 95 wt.-% hydrocarbons having a carbon number in the range from C5 to C10; and
   more than 30 wt.-%, preferably more than 40 wt.-%, more preferably more than 50 wt.-% isoparaffins.
21. The process according to any one of the preceding items, wherein the hydrotreated bio-based hydrocarbon feed has a biogenic carbon content, as determined in accordance with EN 16640 (2017), of more than 50 wt.-%, especially more than 60 wt.-% or more than 70 wt.-%, preferably more than 80 wt.-%, more preferably more than 90 wt.-% or more than 95 wt.-%, even more preferably about 100 wt.-%, based on the total weight of carbon in the hydrotreated bio-based hydrocarbon feed.
22. The process according to any one of the preceding items, wherein the hydrotreated bio-based hydrocarbon feed comprises, based on the total weight of the hydrotreated bio-based hydrocarbon feed, at most 5 wt.-%, preferably at most 3 wt.-%, more preferably at most 2 wt.-%, even more preferably at most 1 wt.-% hydrocarbons having a carbon number of at least C22.
23. The process according to any one of the preceding items, wherein the wt.-% amount of the hydrotreated bio-based hydrocarbon feed in the catalytic cracking feed (catalytic cracking fresh feed, i.e. excluding an optional cracking effluent recycle feed) is more than 80 wt.-%, such as more than 90 wt.-%, preferably more than 95 wt.-%, more preferably at least 99 wt.-%, based on the total weight of the catalytic cracking fresh feed.
24. The process according to any one of the preceding items, wherein the catalytic cracking feed further comprises a cracking effluent recycle feed.
25. The process according to item 24, wherein the wt.-% amount of the cracking effluent recycle feed in the catalytic cracking feed is at least 10 wt.-% or more than 10 wt.-% or more than 20 wt.-% or more than 30 wt.-% or more than 40 wt.-% or more than 50 wt.-% or more than 60 wt.-% or more than 70 wt.-% or more than 80 wt.-% or more than 90 wt.-%, and less than 99 wt.-% or less than 90 wt.-% or preferably at most 80 wt.-% or less than 80 wt.-% or less than 70 wt.-% or less than 60 wt.-% or less than 50 wt.-% or less than 40 wt.-% or less than 30 wt.-% or less than 20 wt.-%, based on the total weight of the catalytic cracking feed, preferably from 10 wt.-% to 80 wt.-%.
26. The process according to item 24 or 25, wherein the sum of the wt.-% amounts of the hydrotreated bio-based hydrocarbon feed and the cracking effluent recycle feed in the catalytic cracking feed is more than 80 wt.-%, such as more than 85 wt.-% or more than 90 wt.-%, preferably more than 95 wt.-% such as more than 97 wt.-%, more preferably at least 99 wt.-%, based on the total weight of the catalytic cracking feed.
27. The process according to any one of items 24 to 26, wherein the weight ratio of the hydrotreated bio-based hydrocarbon feed to the cracking effluent recycle feed (hydrotreated bio-based hydrocarbon feed: cracking effluent recycle feed) in the catalytic cracking feed is at least 10:90, preferably at least 20:80, more preferably at least 50:50, such as at least 80:20.
28. The process according to any one of items 24 to 27, wherein the weight ratio of the hydrotreated bio-based hydrocarbon feed to the cracking effluent recycle feed (hydrotreated bio-based hydrocarbon feed: cracking effluent recycle feed) in the catalytic cracking feed is at most 99:1, such as at most 90:10, preferably at most 80:20, such as at most 50:50, or at most 20:80.
29. The process according to any one of the preceding items, further comprising recovering from the cracking effluent a fraction of hydrocarbons having a carbon number of at least C5, and recycling at least a portion of said fraction to the catalytic cracking feed as a cracking effluent recycle feed.
30. The process according to any one of items 24 to 29, wherein the cracking effluent recycle feed comprises, based on the total weight of the cracking effluent recycle feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C5.
31. The process according to any one of items 24 to 30, wherein the cracking effluent recycle feed comprises, based on the total weight of the cracking effluent recycle feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C11.
32. The process according to any one of items 24 to 31, wherein the cracking effluent recycle feed comprises, based on the total weight of the cracking effluent recycle feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C14.
33. The process according to any one of the preceding items, further comprising recovering from the cracking effluent a fraction rich in aromatics as a bio-aromatics component.
34. The process according to any one of the preceding items, further comprising recovering from the cracking effluent a fraction rich in bio-ethylene as a bio-ethylene composition, preferably comprising more than 50 wt.-% of ethylene, based on the total weight of the bio-ethylene composition.
35. The process according to any one of the preceding items, further comprising recovering from the cracking effluent a fraction rich in C4 hydrocarbons as a bio-C4 composition, preferably comprising more than 50 wt.-% of C4 hydrocarbons, based on the total weight of the bio-C4 composition, such as a fraction rich in C4 olefins as a bio-butylene composition, preferably comprising more than 50 wt.-% of C4 olefins, based on the total weight of the bio-butylene composition.
36. The process according to any one of the preceding items, wherein the recovering, especially in step (D), comprises one or more of distilling, fractionating, separating, evaporating, flash-separating, membrane separating, extracting, using extractive-distillation, using chromatography, using molecular sieve adsorbents, using thermal diffusion, complex forming, crystallizing.
37. The process according to any one of the preceding items, wherein the recovering, especially in step (D), comprises at least one or more of fractionating, distilling, extracting, and using extractive-distillation.
38. The process according to any one of the preceding items, wherein the recovering comprises at least fractionating.
39. The process according to any one of the preceding items, wherein the step (D) further comprises recovering from the cracking effluent a fraction rich in C5-C10 hydrocarbons as a bio-gasoline component.
40. A bio-propylene composition comprising bio-propylene and bio-propane, wherein the total content of the bio-propylene is at least 80 wt.-%, based on the total weight of the bio-propylene composition, and the weight ratio of bio-propylene to bio-propane is at least 4.5.
41. The bio-propylene composition according to item 40, wherein the total content of the bio-propylene is at least 85 wt.-%, based on the total weight of the bio-propylene composition, and the weight ratio of bio-propylene to bio-propane is at least 5.3.
42. The bio-propylene composition according to item 40 or 41, wherein the total content of the bio-propylene is at least 90 wt.-%, such as at least 99 wt.-%, based on the total weight of the bio-propylene composition, and the weight ratio of bio-propylene to bio-propane is at least 9.0.
43. The bio-propylene composition according any one of items 40 to 42, wherein the bio-propylene composition is obtainable by the process according to any one of items 1 to 39.
44. A method for producing a (co)polymer composition comprising:
   producing a bio-propylene composition according to the process of any one of items 1 to 39, optionally purifying the bio-propylene composition, and/or optionally derivatising at least a part of the bio-propylene molecules in the optionally purified bio-propylene composition to obtain a polymerizable composition of bio-monomer(s), and (co)polymerizing a monomer composition comprising the polymerizable composition of bio-monomers to obtain the (co)polymer composition.
45. The method according to item 44, wherein the polymerizable composition of bio-monomer(s) comprises or consists of olefinically unsaturated bio-monomers or epoxide bio-monomers.
46. The method according to item 44 or 45, wherein the polymerizable composition of bio-monomer(s) comprises or consists of at least one olefinically unsaturated bio-monomer selected from the group consisting of bio-propylene, bio-acrylic acid, bio-acrylonitrile, and bio-acrolein, or at least one epoxide bio-monomer selected from the group consisting of bio-propylene oxide.
47. The method according to any one of items 44 to 45, wherein the monomer composition further comprises other (co)monomer(s) and/or additive(s).
48. A (co)polymer composition obtainable by the method according to any one of claims 44 to 47.
49. A bio-gasoline component comprising at least 75 wt.-% C5-C10 hydrocarbons; at least 8 wt.-% cyclic hydrocarbons; n-paraffins, and at least 7 wt.-% isoparaffins; and wherein the sum of the wt.-% amounts of isoparaffins and n-paraffins in the bio-gasoline component is at most 65 wt.-%, based on the total weight of the bio-gasoline component.
50. The bio-gasoline component according to item 49, comprising at least 85 wt.-%, more preferably at least 90 wt.-% C5-C10 hydrocarbons.
51. The bio-gasoline component according to item 49 or 50, comprising at least 10 wt.-%, more preferably at least 15 wt.-% cyclic hydrocarbons.
52. The bio-gasoline component according to any one of items 49 to 51, comprising at least 12 wt.-%, more preferably at least 20 wt.-% iso-paraffins.
53. The bio-gasoline component according to any one of items 49 to 52, wherein the sum of the wt.-% amounts of isoparaffins and n-paraffins in the bio-gasoline component is at most 60 wt.-%, more preferably at most 55 wt.-%; based on the total weight of the bio-gasoline component.
54. The bio-gasoline component according to any one of items 49 to 53, wherein the bio-gasoline component is obtainable by the process according to item 39.
55. The bio-gasoline component according to any one of items 49 to 54, having a RON value of at least 60.
56. The bio-gasoline component according to any one of items 49 to 55, having a MON value of at least 50.
57. The bio-gasoline component according to any one of items 49 to 56, having a RON minus MON value of at least 5.
58. The bio-gasoline component according to any one of items 49 to 57, having a 5% boiling point of 50° C. or more and a 95% boiling point of 220° C. or less, as determined in accordance with ENIS03405.
59. The bio-gasoline component according to any one of items 49 to 58, comprising at most 1 wt.-% benzene.
60. The bio-gasoline component according to any one of items 49 to 59, comprising at most 1 wt.-% total aromatics, preferably at most 0.01 wt.-% total aromatics.

### BRIEF DESCRIPTION OF DRAWINGS

Some example embodiments will be described with reference to the accompanying figures, in which:

FIG. 1 is a simplified flow diagram of FCC reactor system usable in embodiments herein;

FIG. 2 shows selected characteristics of a cracking effluent stream or a specified fraction thereof as a function of cracking feed isoparaffin content (wt.-%);

FIG. 3 shows a schematic diagram of a steam cracking apparatus disclosed in the prior art (WO 2020/201614 A1).

### DETAILED DESCRIPTION

All standards referred to herein are the latest revisions available on Oct. 31, 2020, unless otherwise mentioned.

All embodiments (such as all preferred values and/or ranges within the embodiments) of the present invention may be combined with each other to give new (preferred) embodiments, unless explicitly specified otherwise or unless such a combination would result in a contradiction.

Conversion refers in the context of the present disclosure to the wt:wt ratio of the compounds split in the catalytic cracking into compounds having a smaller carbon number (converted feed) to the catalytic cracking feed subjected to the catalytic cracking (weight of converted feed:weight of feed subjected to cracking).

Conversion normalized yield, sometimes simply referred to as productivity, refers herein to a yield expressed as weight of a certain compound or certain compounds in a cracking effluent stream normalized by the weight of the converted catalytic cracking feed, i.e. weight of a certain compound or certain compounds in a cracking effluent/weight of converted catalytic cracking feed. The conversion normalized yield may be expressed as weight percentage, namely 100%×(weight of a certain compound or certain compounds in a cracking effluent/weight of converted catalytic cracking feed).

In the present disclosure, the term “gaseous compounds (NTP)” refers to compounds that are gaseous at normal temperature and pressure (20° C. and 101.325 kPa).

In the present disclosure, the terms “cracking effluent” and “catalytic cracking effluent” each refer to the effluent of a catalytic cracking reactor (more specifically of the catalytic cracking reactor of step (C)), but excluding the catalyst and the coke discharged from the catalytic cracking reactor.

It is generally known that alkane and paraffin are synonyms and can be used interchangeably. Isoparaffins (i-paraffins) are branched, open chain saturated hydrocarbons, and normal paraffins (n-paraffins) are unbranched linear, saturated hydrocarbons. In the context of the present disclosure, the term “paraffin” refers to n-paraffins and isoparaffins. Similarly, the term “paraffinic” refers herein to compositions comprising n-paraffins and/or isoparaffins.

In certain embodiments, the isoparaffins have one or more C1-C9, typically C1-C2, alkyl side chains (i.e. side chains having 1 to 9, typically 1 to 2 carbon atoms). Preferably, the side chains are methyl side chains, and the isoparaffins are mono-, di-, tri- and/or tetramethyl substituted.

Further, cyclic saturated hydrocarbons are designated as naphthenes in the present disclosure, and hydrocarbons containing at least one cyclic structure having delocalized, alternating pi bonds all the way around the cyclic structure are designated as aromatics. Non-limiting examples of the aromatics include so-called BTX, i.e. benzene, toluene and xylenes, and also condensed aromatic ring compounds and aromatic olefins (e.g. styrene). Combined naphthenes and aromatics are jointly designated as cyclic hydrocarbons (or cyclics). Furthermore, unsaturated hydrocarbons, alkenes, containing one or more carbon atoms linked by a double or triple bond, excluding aromatics, are designated as olefins in the present disclosure.

In the present context, the term “bio-based” or “bio-” refers to a material which is derived from renewable sources (as opposed to fossil sources) in full or in part. Carbon atoms of renewable or biological origin comprise a higher number of unstable radiocarbon (¹⁴C) atoms compared to carbon atoms of fossil origin. Therefore, it is possible to distinguish between carbon compounds derived from renewable or biological sources or raw material and carbon compounds derived from fossil sources or raw material by analysing the ratio of ¹²C and ¹⁴C isotopes. Thus, a particular ratio of said isotopes can be used as a “tag” to identify renewable carbon compounds and differentiate them from non-renewable carbon compounds. The isotope ratio does not change in the course of chemical reactions. Examples of a suitable method for analysing the content of carbon from biological or renewable sources are DIN 51637 (2014), ASTM D6866 (2020) and EN 16640 (2017). As used herein, the content of carbon from biological or renewable sources is expressed as the biogenic carbon content meaning the amount of biogenic carbon in the material as a weight percent of the total carbon (TC) in the material (in accordance with ASTM D6866 (2020) or EN 16640 (2017)). In the present context, the term “bio-based” or “bio-” preferably refers to a material having a biogenic carbon content of more than 50 wt.-%, especially more than 60 wt.-% or more than 70 wt.-%, preferably more than 80 wt.-%, more preferably more than wt.-% or more than 95 wt.-%, even more preferably about 100 wt.-%, based on the total weight of carbon in the material (EN 16640 (2017)).

The present disclosure provides a process for manufacturing a bio-propylene composition. The process may further optionally provide, among others, a bio-gasoline component and/or a bio-aromatics component.

Specifically the present disclosure relates to a process for manufacturing a bio-propylene composition, and optionally a bio-gasoline component, the process comprising the following steps (A) to (D):
(A) hydrotreating an oxygen-containing bio-based feedstock to obtain a hydrotreatment effluent comprising oxygen-depleted hydrocarbons, and subjecting the hydrotreatment effluent to a gas-liquid separation, and optionally to a fractionation, to provide a hydrotreated bio-based hydrocarbon feed containing less than 1.0 wt.-%, preferably less than 0.8 wt.-%, more preferably less than 0.5 wt.-%, of gaseous compounds (NTP);
(B) providing a catalytic cracking feed comprising the hydrotreated bio-based hydrocarbon feed;
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in propylene as the bio-propylene composition, and optionally a fraction rich in C5-C10 hydrocarbons as the bio-gasoline component.

In another aspect, the present disclosure relates to a process for manufacturing a bio-propylene composition, and optionally a bio-gasoline component, the process comprising the following steps (B′), (C) and (D):
(B′) providing a catalytic cracking feed comprising a hydrotreated bio-based hydrocarbon feed containing less than 1.0 wt.-%, preferably less than 0.8 wt.-%, more preferably less than 0.5 wt.-%, of gaseous compounds (NTP);
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in propylene as the bio-propylene composition, and optionally a fraction rich in C5-C10 hydrocarbons as the bio-gasoline component.

The process of the present disclosure is a particularly favourable integrated process for producing high-value bio-based hydrocarbons, and especially bio-propylene. By combining catalytic cracking using a moving solid catalyst (MSC), especially fluidized solid catalyst, in combination with a hydrotreated bio-based hydrocarbon feed containing less than 1.0 wt.-% of gaseous compounds (NTP), it is possible to provide a bio-based cracking effluent having a favourable product distribution and especially high share of bio-propylene. Also other valuable cracking products are obtainable, especially a bio-gasoline component, and also bio-aromatics. The process of the present disclosure can be integrated into existing petrochemical production lines, since the equipment for catalytic cracking using moving solid catalyst already exist. One example of these processes is FCC (Fluid Catalytic Cracking) which is commonly employed in petrochemical processes. Accordingly, there is no need to set up new equipment and, consequently, it is easily possible to increase the sustainability of a petrochemical plant using minimum effort.

Steam cracking is the number one process for manufacturing propylene from fossil-based feedstocks, although it is only obtained as a by-product to fossil ethylene. On top of that steam cracking is also one of the most energy-intensive industrial processes. The present process greatly alleviates these drawbacks as allowing propylene production as the main product, in greater amounts, and using far less energy.

In particular, the process of the present disclosure involving catalytic cracking of a hydrotreated bio-based hydrocarbon feed containing less than 1 wt.-% of gaseous compounds (NTP), using a moving solid catalyst, provides surprisingly high propylene (C3=) productivity compared to steam cracking (SC) which is the current industry standard for propylene manufacturing. In addition, a high propylene to ethylene (C3=/C2=) weight ratio is achieved, typically more than 2.0, while in SC the C3=/C2=ratio is typically far below 1. This is especially beneficial as using currently available technologies it is more difficult to produce propylene from bio-based raw materials, than ethylene. Surprisingly, the process of the present disclosure facilitates easy recovery of a bio-propylene composition having high bio-propylene content and very low bio-propane content, and thus excellent propylene/total C3 (propylene/{summed amount of propylene and propane}) shares are obtainable, typically exceeding 80 wt.-%. Moreover, it was surprisingly found out that when using increasing isoparaffin content in the feed, productivity of propane decreases compared to the productivity of propylene. So when employing a high isoparaffin content feed, further improved propylene/total C3 shares are achievable, even reaching 90 wt.-%, which is highly advantageous as explained in the following.

Since propane and propylene have similar molecular size and physical properties, their separation is challenging. This separation is mostly carried out in distillation columns generally having more than 150 theoretical plates, and operating with very high reflux ratios, often 10-20, and at a high pressure, typically of about 16-26 atm. The process requires high capital cost and very high energy consumption. The propylene purity will affect the grade and value of the propylene product: for refinery grade 50-70% purity may suffice, while for chemical grade 90-95 purity is typically required, and for polymer grade even 99.5% purity or higher. A typical setting for obtaining the polymer grade purity involves using a distillation column, also called a “splitter”, constituted by 152 theoretical plates and operating initially with a reflux of 24.1 in order to separate propane/propylene mixture until the purity of 99.5% is obtained. Logically, the higher the propylene/total C3 share in the beginning of the propane/propylene separation, the lower the energy needed for reaching the polymer grade purity. Also less complex/expensive equipment may suffice. In addition to the high productivity of propylene and low productivity of propane, it is important that the hydrotreated bio-based hydrocarbon feed fed to the catalytic cracking reactor contains less than 1.0 wt.-% of gaseous compounds (NTP), i.e. compounds that are gaseous at normal temperature and pressure (20° C., 101.325 kPa), to ensure that the hydrotreated bio-based hydrocarbon feed contains at most very low amounts of propane, so that it is not carried over unconverted to the cracking effluent thereby decreasing the propylene/total C3 share in the cracking effluent. Limiting the amount of gaseous compounds (NTP) is especially important when the hydrotreated bio-based hydrocarbon feed has been obtained by hydrotreating fatty acid glycerides containing bio-based feedstock, as this causes formation of elevated amounts of propane during the hydrotreatment. Additionally, during hydrotreatment of an oxygen-containing bio-based feedstock especially CO and CO₂, but also NH₃ and/or H₂S gases, are formed, these causing catalyst fouling and/or deactivation of active sites of the cracking catalyst. The hydrotreatment effluent may contain also residual molecular hydrogen, which, if carried over to the catalytic cracking reactor, may decrease the bio-propylene yield. So also for these reasons it is important to use gas-depleted hydrotreated bio-based hydrocarbon feed. Catalytic cracking of the specified feed using the present process may achieve 90% purity by simply fractionating C3 hydrocarbon fraction from the cracking effluent, which may suffice for chemical grade propylene, and thus a dedicated propane/propylene separation may not be needed at all.

In addition to the easy recovery of a bio-propylene composition, the present process allows also recovery of high quality bio-gasoline component. Bio-gasoline components have been successfully manufactured as a by-product to renewable diesel by hydrotreating vegetable and/or animal oils (so-called HVO technology). However, these bio-gasoline components can be used only in limited amounts in gasoline blends due to their high paraffinicity and low content of cyclic hydrocarbons, and low octane. The bio-gasoline components obtainable by the present process have higher content of cyclic hydrocarbons, allowing higher blending ratio in gasoline blends, compared to a bio-gasoline component obtainable by an HVO process.

The productivity of the bio-gasoline component, i.e. a fraction rich in C5-C10 hydrocarbons, such as hydrocarbons from carbon chain length C5 to hydrocarbons boiling at about 221° C., may be further increased by increasing the isoparaffin content in the hydrotreated bio-based hydrocarbon feed. Also the properties desired for gasoline compositions, including RON and MON values, improve along increasing isoparaffin content of the feed, so usability of the bio-gasoline component in gasoline blends is enhanced. An increasing isoparaffins content in the hydrotreated bio-based hydrocarbon feed improves also productivity of cyclic hydrocarbons, including bio-aromatics, especially BTX (benzene, toluene, xylene), and naphthenes. When present in the bio-gasoline component, the cyclic hydrocarbons, i.e. naphthenes and aromatics, improve its octane rating. Still, the bio-gasoline component obtainable by the present process contains far less aromatics than fossil-based gasoline compositions making it less hazardous for health. The bio-gasoline component may also be used in chemical products intended for use by industry or households, such as in solvents, thinners and spot removers.

Additionally, an increasing isoparaffins content in the hydrotreated bio-based hydrocarbon feed decreases total C4 hydrocarbon yield in the cracking effluent, which is beneficial because C4 utilization as such e.g. in gasoline compositions is very limited. In the present context total C4 hydrocarbons means both C4 paraffins and C4 olefins, and C4 olefins cover 1-butene, trans-2-butene, cis-2-butene, butadiene, isobutene.

Furthermore, catalytic cracking of the specified hydrotreated bio-based hydrocarbon feed using the present process generates just a fraction of the methane emissions of steam cracking. The present process is therefore highly beneficial since methane is a low-value product, and a strong greenhouse gas. The present process thus further contributes to improved sustainability without need for expensive new equipment.

Finally, in typical industrial scale processes it is usually desired to keep the productivity of the main product constant. With the present process it is possible to maintain bio-propylene productivity approximately constant, and adjust the productivity of the further products such as the bio-gasoline component and the bio-aromatics e.g. depending on their market demand or price, in a simple manner, by varying the isoparaffin content of the feed.

The present process can be fully integrated into a conventional petrochemical process in accordance with availability of the hydrotreated bio-based hydrocarbon feed. As a matter of course, the more hydrotreated bio-based hydrocarbon feed is employed, the higher the sustainability of the overall process. Similarly, the beneficial influence of the specified hydrotreated bio-based hydrocarbon feed on the composition of the cracking effluent and/or distribution of the catalytic cracking products therein will be more pronounced the higher the share of the hydrotreated bio-based hydrocarbon feed in the catalytic cracking fresh feed (i.e. the part of the catalytic cracking feed other than an optional cracking effluent recycle feed, if present). Nevertheless, it is believed that already low amounts of the hydrotreated bio-based hydrocarbon feed in the catalytic cracking feed have a beneficial influence on the distribution of the catalytic cracking products in the cracking effluent.

Preparation of the Hydrotreated Bio-Based Hydrocarbon Feed

The present disclosure provides a process for catalytically cracking a catalytic cracking feed comprising a hydrotreated bio-based hydrocarbon feed containing less than 1 wt.-%, preferably less than 0.8 wt.-%, more preferably less than 0.5 wt.-%, of gaseous compounds (NTP). Generally the catalytic cracking feed may comprise a hydrotreated bio-based hydrocarbon feed prepared by any method as long as it contains less than 1.0 wt.-% of gaseous compounds (NTP).

Preferably the specified hydrotreated bio-based hydrocarbon feed is prepared by (A) hydrotreating an oxygen-containing bio-based feedstock to obtain a hydrotreatment effluent comprising oxygen-depleted hydrocarbons, and subjecting the hydrotreatment effluent to a gas-liquid separation, and optionally to a fractionation. In the present disclosure, an oxygen-containing bio-based feedstock refers to oxygen-containing bio-based feedstock having a biogenic carbon content of more than 50 wt.-%, especially more than 60 wt.-% or more than 70 wt.-%, preferably more than 80 wt.-%, more preferably more than 90 wt.-% or more than 95 wt.-%, even more preferably about 100 wt.-%, based on the total weight of carbon in the oxygen-containing bio-based feedstock (EN 16640 (2017)). In the present disclosure, a hydrotreatment effluent comprising oxygen-depleted hydrocarbons refers to a hydrotreatment effluent comprising at most 3 wt.-% oxygen calculated as elemental 0.

The oxygen-containing bio-based feedstock used in the process of the present disclosure may be any oxygen-containing bio-based organic material capable of being deoxygenated by hydrotreating.

In certain embodiments in step (A) the oxygen-containing bio-based feedstock comprises one or more of fatty acids, fatty acid esters, resin acids, resin acid esters, sterols, fatty alcohols, oxygenated terpenes. More specifically, examples of oxygen-containing compounds in the bio-based feedstock include fatty acids, whether in free or salt form; fatty acid esters, such as mono-, di- and triglycerides, alkyl esters such as methyl or ethyl esters, etc; resin acids, whether in free or salt form; resin acid esters, such as alkyl esters, sterol esters etc; sterols; fatty alcohols; oxygenated terpenes; and other organic acids, ketones, alcohols, and anhydrides.

In certain embodiments in step (A) the oxygen-containing bio-based feedstock comprises one or more of vegetable oils, animal fats, microbial oils, thermally and/or enzymatically liquefied biomass. More specifically, examples of oxygen-containing bio-based feedstock include vegetable oils such as rapeseed oil, canola oil, soybean oil, coconut oil, sunflower oil, palm oil, palm kernel oil, peanut oil, linseed oil, sesame oil, maize oil, poppy seed oil, cottonseed oil, soy oil, tall oil, corn oil, castor oil, jatropha oil, jojoba oil, olive oil, flaxseed oil, camelina oil, safflower oil, babassu oil, seed oil of any of Brassica species or subspecies, such as Brassica carinata seed oil, Brassica juncea seed oil, Brassica oleracea seed oil, Brassica nigra seed oil, Brassica napus seed oil, Brassica rapa seed oil, Brassica hirta seed oil and Brassica alba seed oil, and rice bran oil, or fractions or residues of said vegetable oils such as palm olein, palm stearin, palm fatty acid distillate (PFAD), purified tall oil, tall oil fatty acids, tall oil resin acids, distilled tall oil, tall oil unsaponifiables, tall oil pitch (TOP), and used cooking oil of vegetable origin; animal fats such as tallow, lard, yellow grease, brown grease, fish fat, poultry fat, and used cooking oil of animal origin; microbial oils, such as algal lipids, fungal lipids and bacterial lipids; thermally and/or enzymatically liquefied biomass such as pyrolyzed biomass, hydrothermally liquefied biomass, solvothermally liquefied biomass and/or enzymatically hydrolysed biomass. Preferably the oxygen-containing bio-based feedstock comprises one or more of vegetable oils, animal fats, and microbial oils, as hydrotreating these feedstocks results in mainly paraffinic hydrocarbons, favouring bio-propylene generation in the catalytic cracking step (C).

Hydrotreating the oxygen-containing bio-based feedstock comprises preferably deoxygenation. Deoxygenation means removal of oxygen as H₂O, CO₂ or CO from the oxygen containing hydrocarbons by hydrodeoxygenation, decarboxylation and/or decarbonylation.

Hydrotreating may involve various reactions where molecular hydrogen reacts with other components, or components undergo molecular conversions in the presence of molecular hydrogen and a catalyst. These reactions include but are not limited to hydrogenation, hydrodeoxygenation, hydrodesulphurization, hydrodenitrogenation, hydrodemetallization, hydrocracking, hydropolishing, hydroisomerization and hydrodearomatization.

Preferably hydrotreating comprises deoxygenation and isomerization reactions. More preferably hydrotreating comprises deoxygenation by hydrodeoxygenation (HDO) and isomerization by hydroisomerization. Hydrodeoxygenation means removal of oxygen as H₂O from the oxygen containing hydrocarbons by means of molecular hydrogen under influence of a catalyst, while hydroisomerization means formation of branches to the hydrocarbons by means of molecular hydrogen under influence of a catalyst that can be same or different as for HDO.

In certain embodiments in step (A) hydrotreating comprises at least deoxygenation and isomerization, preferably hydrodeoxygenation and isomerization.

In certain embodiments the deoxygenation comprises hydrodeoxygenation, decarboxylation and/or decarbonylation.

In certain embodiments the hydrodeoxygenation and isomerization are conducted simultaneously in the same hydrotreating step, and/or separately in subsequent hydrotreating steps.

In certain embodiments in step (A) hydrotreating is conducted in two or more subsequent hydrotreating steps, preferably a first hydrotreating step comprising at least hydrodeoxygenation; and a second hydrotreating step comprising at least isomerization.

In certain embodiments in step (A) hydrotreating comprises at least deoxygenation, preferably hydrodeoxygenation, in the presence of a hydrocarbon diluent, preferably a recycled fraction of hydrotreatment effluent. In this way the temperature can be better controlled during the hydrotreating thus reducing production of side products (such as gases) and the overall productivity of products having favourable properties in the catalytic cracking is further improved.

A hydrotreated bio-based hydrocarbon feed containing less than 1.0 wt.-%, preferably less than 0.8 wt.-%, more preferably less than 0.5 wt.-%, of gaseous compounds (NTP) usable in the present process may also be prepared e.g. by gasification of biomass, such as (ligno)cellulosic biomass and/or organic municipal solid waste, to obtain syngas, subjecting at least part of the syngas to Fischer-Tropsch reaction conditions, in the presence of molecular hydrogen and a metal catalyst, to obtain n-paraffins, followed by hydrotreatment comprising hydroisomerization and/or hydrocracking to obtain a hydrotreatment effluent comprising isoparaffins, and subjecting the hydrotreatment effluent to a gas-liquid separation. Alternatively, a hydrotreated bio-based hydrocarbon feed may be provided by a route other than a Fischer-Tropsch process and a Fischer-Tropsch-based hydrocarbon feed may be used as a co-feed in the catalytic cracking step (C).

Many conditions for hydrotreatment/hydrodeoxygenation are known to the skilled person. The hydrotreatment of an oxygen-containing bio-based feedstock in accordance with the present disclosure may be carried out in the presence of a sulphided metal catalyst. The metal can be one or more Group VI metals, such as Mo or W, or one or more Group VIII non-noble metals such as Co or Ni. The catalyst may be supported on any conventional support, such as alumina, silica, zirconia, titania, amorphous carbon, molecular sieves or combinations thereof. Usually the metal will be impregnated or deposited on the support as metal oxides. They will then typically be converted into their sulphides. Examples of typical catalysts for hydrodeoxygenation are molybdenum containing catalysts, NiMo, CoMo, or NiW catalysts; supported on alumina or silica, but many other hydrodeoxygenation catalysts are known in the art and have been described together with or compared to NiMo and/or CoMo catalysts. The hydrodeoxygenation is preferably carried out under the influence of sulphided NiMo or sulphided CoMo catalysts in the presence of hydrogen gas since these catalysts have been found to provide a good balance between catalyst life and efficiency.

As an alternative catalyst, Pt and/or Pd catalysts supported on a conventional support (e.g. those indicated above) may be employed. On the other hand, sulphided metal catalysts are preferred.

The hydrotreatment may be performed under a hydrogen pressure from 1 to 200 bar (absolute), preferably from 10 to 150 bar, from 10 to 100 bar, from 30 to 100 bar, or from 30 to 70 bar, at temperatures from 200 to 500° C., preferably from 200 to 400° C., from 230 to 400° C., from 230 to 370° C., or from 280 to 370° C., and liquid hourly space velocities of 0.1 h⁻¹ to 3.0 h⁻¹, preferably of 0.2 to 2.0 h⁻¹.

By feeding hydrogen (H₂) to hydrogenation so as to provide a (H₂ partial) pressure in the above-mentioned ranges, efficient HDO (hydrodeoxygenation), HDN (hydrodenitrogenation), and HDS (hydrodesulphurisation) reactions can be ensured while controlling (thermal) cracking reactions at a low level.

During the hydrotreatment step (A) using a sulphided catalyst, the sulphided state of the catalyst is preferably maintained by addition of a sulphur-containing compound to the oxygen-containing bio-based feedstock and/or to the diluent and/or fed along the hydrogen gas and/or separately to the hydrotreatment reactor. Usually, the sulphur is added in the form of H₂S, but it is nevertheless possible to add the sulphur in the form of other sulphur compounds such as sulphides, disulphides (e.g. dimethyl disulphide, DMDS), polysulphides, thiols, thiophene, benzothiophene, dibenzothiophene and derivatives thereof, as a single compound or a mixture of two or more types of these compounds. It is also possible to blend a sulphur containing mineral oil diluent with the oxygen-containing bio-based feedstock.

The hydrotreated bio-based hydrocarbon feed used in the process of the present disclosure may be provided by subjecting at least a portion of n-paraffins formed in a deoxygenation (preferably hydrodeoxygenation) step to an isomerisation treatment to form isoparaffins. The isomerisation treatment is not particularly limited. Nevertheless, catalytic isomerisation treatments are preferred. Typically, subjecting n-paraffins formed in a hydrotreatment step from an oxygen-containing bio-based feedstock to an isomerisation treatment forms predominantly methyl substituted isoparaffins. The severity of isomerization conditions and choice of catalyst controls the amount of methyl branches formed and their distance from each other in the carbon backbone. The isomerization step may comprise further intermediate steps such as a purification step and a fractionation step. Purification and/or fractionation steps allows better control of the properties of the hydrotreatment effluent being formed.

The isomerization treatment is preferably performed at a temperature selected from the range 200 to 500° C., preferably 280 to 400° C., and at a pressure selected from the range 20-150 bar (absolute), preferably 30-100 bar. The isomerization treatment may be performed in the presence of known isomerization catalysts, for example, catalysts containing a molecular sieve and/or a metal selected from Group VIII of the Periodic Table and a carrier. Preferably, the isomerization catalyst is a catalyst containing SAPO-11 or SAPO-41 or ZSM-22 or ZSM-23 or ferrierite and Pt, Pd, or Ni and Al₂O₃ or SiO₂. Typical isomerisation catalysts are, for example, Pt/SAPO-11/Al₂O₃, Pt/ZSM-22/Al₂O₃, Pt/ZSM-23/Al₂O₃ and/or Pt/SAPO-11/SiO₂. Catalyst deactivation may be reduced by the presence of molecular hydrogen in the isomerisation treatment. Therefore, the presence of added hydrogen in the isomerisation treatment is preferred. In certain embodiments, the hydrotreatment catalyst(s) and the isomerization catalyst(s) are not in contact with the reaction feed (the oxygen-containing bio-based feedstock and/or n-paraffins and/or i-paraffins derived therefrom) at the same time. For example, the hydrotreatment and the isomerisation treatment are conducted in separate reactors, or carried out separately.

In some cases, it may be favourable that only a portion of the n-paraffins formed in the hydrotreatment step is subjected to an isomerization treatment. A portion of the n-paraffins formed in the hydrotreatment step may be separated, the separated n-paraffins then subjected to the isomerisation treatment to form isoparaffins. After being subjected to the isomerisation treatment, the separated (and isomerized) paraffins are optionally re-unified with the remainder of the paraffins. Alternatively, all of the n-paraffins formed in the hydrotreatment step may be subjected to the isomerization treatment.

Incidentally, the isomerisation treatment (when carried out as a separate step, i.e. not simultaneously with deoxygenation) is a step which predominantly serves to isomerise the paraffins of the renewable isomeric paraffin composition. While thermal or catalytic conversions (such as hydrotreatment comprising HDO) may result in a minor degree of isomerisation (usually less than 5 wt.-%), the isomerisation step which may be employed in the present process is the step which leads to a significant increase in the isoparaffin content of the hydrotreatment effluent.

In certain embodiments the oxygen-containing bio-based feedstock may be subjected to a pre-treatment for reducing contaminants prior to the hydrotreating. The pre-treatment may comprise reducing contaminants containing S, N and/or P and/or metal-containing contaminants in the oxygen-containing bio-based feedstock. For example, the pre-treatment may comprise one or more selected from washing, degumming, bleaching, distillation, fractionation, rendering, heat treatment, evaporation, filtering, adsorption, partial hydrodeoxygenation, full or partial hydrogenation, centrifugation or precipitation, hydrolysis and transesterification. The pretreatment may enhance significantly the hydrotreatment catalyst activity and lifetime, thereby beneficially contributing to the composition and quality of the hydrotreatment effluent.

In certain embodiments the hydrotreatment effluent subjected to a gas-liquid separation comprises combined effluent from two or more different hydrotreating steps of same step (A); or from two or more different steps (A) hydrotreating oxygen-containing bio-based feedstocks.

In certain embodiments in step (A) subjecting the hydrotreatment effluent to a gas-liquid separation removes at least part of C1-C3 hydrocarbons, Hz, etc. to provide the hydrotreated bio-based hydrocarbon feed containing less than 1.0 wt.-% of gaseous compounds (NTP).

In the gas-liquid separation, the amount of gaseous compounds (NTP) in the hydrotreatment effluent is decreased at least to the specified level. The gas-liquid separation step may be carried out as a separate step (after the effluent has left the hydrotreatment reactor or reaction zone) and/or as an integral step of the hydrotreatment step, e.g. within the hydrotreatment reactor or reaction zone. Majority of the water that may form during HDO and potentially carried-over from the fresh oxygen-containing bio-based feedstock may be removed for example via a water boot in the gas-liquid separation step.

In various embodiments the gas-liquid separation is carried out at a temperature of 0° C. to 500° C., such as 15° C. to 300° C., or 15° C. to 150° C., preferably 15° C. to such as 20° C. to 60° C., and preferably at the same pressure as that of the hydrotreatment step. In general, the pressure during the gas-liquid separation step may be 1-200 bar (gauge), preferably 10-100 bar (gauge), or 30-70 bar (gauge).

It is important that the hydrotreated bio-based hydrocarbon feed contains less than 1 wt.-%, preferably less than 0.8 wt.-%, more preferably less than 0.5 wt.-%, of gaseous compounds (NTP), i.e. compounds that are gaseous at normal temperature and pressure. This is to ensure that the hydrotreated bio-based hydrocarbon feed contains at most very low amounts of propane, so that it is not carried over unconverted to the cracking effluent thereby decreasing the propylene/total C3 share in the cracking effluent. Limiting the amount of gaseous compounds (NTP) is especially important when the hydrotreated bio-based hydrocarbon feed has been obtained by hydrotreating bio-based feedstock containing fatty acid glycerides, as this causes formation of elevated amounts of propane during the hydrotreatment. Furthermore, as light hydrocarbon gases, including propane and butane, require more severe reaction conditions than longer hydrocarbons, their presence in higher amounts would decrease the propylene yield. Additionally, during hydrotreatment of an oxygen-containing bio-based feedstock CO, CO₂, NH₃, and/or H₂S gases are formed, these causing catalyst fouling and/or deactivation of active sites of the cracking catalyst. The hydrotreatment effluent may contain also residual molecular hydrogen, which, if carried over to the catalytic cracking reactor, may decrease the bio-propylene yield. So also for these reasons it is important to use the specified gas-depleted hydrotreated bio-based hydrocarbon feed. Catalytic cracking of the specified feed using the present process may achieve 90% purity by simply fractionating C3 hydrocarbon fraction from the cracking effluent, which may suffice for chemical grade propylene, and thus a dedicated propane/propylene separation may not be needed at all.

In certain embodiments step (A) further comprises subjecting the hydrotreatment effluent and/or the hydrotreated bio-based hydrocarbon feed containing less than 1.0 wt.-% of gaseous compounds (NTP) to a fractionation. In this way a hydrotreated bio-based hydrocarbon feed containing less than 1.0 wt.-% of gaseous compounds (NTP) and comprising a desired carbon number distribution, for example more than 50 wt.-%, preferably more than 60 wt.-%, further preferably more than 70 wt.-%, more preferably more than 80 wt.-%, and even more preferably more than 90 wt.-%, based on the total weight of the hydrotreated bio-based hydrocarbon feed, hydrocarbons having a carbon number of at least C11 or a carbon number of at least C14, and/or at most 5 wt.-%, preferably at most 3 wt.-%, more preferably at most 2 wt.-%, even more preferably at most 1 wt.-% hydrocarbons having a carbon number of at least C22.

### The Hydrotreated Bio-Based Hydrocarbon Feed

The present disclosure provides a process for catalytically cracking a catalytic cracking feed comprising a hydrotreated bio-based hydrocarbon feed containing less than 1.0 wt.-% of gaseous compounds (NTP).

In certain embodiments, in step (B/B′), the hydrotreated bio-based hydrocarbon feed comprises isoparaffins.

In certain embodiments, in step (B/B′), the hydrotreated bio-based hydrocarbon feed comprises isoparaffins and n-paraffins and the sum of the wt.-% amounts of isoparaffins and n-paraffins in the hydrotreated bio-based hydrocarbon feed is at least 40 wt.-%, preferably more than 50 wt.-%, such as more than 60 wt.-%, more preferably more than 70 wt.-%, such as more than 80 wt.-%, particularly more than 90 wt.-% or even more than 95 wt.-%, based on the total weight of the hydrotreated bio-based hydrocarbon feed. High paraffinicity of the feed enhances the conversion to bio-propylene.

In certain embodiments, in step (B/B′), the hydrotreated bio-based hydrocarbon feed comprises, based on the total weight of the hydrotreated bio-based hydrocarbon feed, less than 25 wt.-% (total) aromatics (aromatics are also called bio-aromatics when produced according to the process of the invention), preferably less than 15 wt.-%, more preferably less than 5 wt.-%, most preferably less than 1 wt.-% (total) bio-aromatics. Aromatics are coke precursors, and coke-formation is beneficial for the energy-efficiency of the present process. However, it is also beneficial that less of the valuable hydrotreated bio-based hydrocarbon feed is lost as coke, and therefore hydrotreated bio-based hydrocarbon feeds containing less bio-aromatics are preferred.

In certain embodiments, in step (B/B′), the hydrotreated bio-based hydrocarbon feed comprises, based on the total weight of the hydrotreated bio-based hydrocarbon feed, more than 1 wt.-% isoparaffins, preferably more than 4 wt.-%, such as more than 5 wt.-%, more preferably more than 30 wt.-%, such as more than 40 wt.-% or more than 50 wt.-% or more than 60 wt.-%, even more preferably more than 70 wt.-%, such as 80 wt.-%, particularly more than 85 wt.-% isoparaffins. Elevated isoparaffin content in the hydrotreated bio-based hydrocarbon feeds is desired as providing plurality of benefits, including enhancing propylene/total C3 ratio, productivity of bio-aromatics, and productivity and quality of the bio-gasoline component in the catalytic cracking.

In certain embodiments, in step (B/B′), the hydrotreated bio-based hydrocarbon feed comprises, based on the total weight of the hydrotreated bio-based hydrocarbon feed, less than 80 wt.-% naphthenes, preferably less than 50 wt.-%, such as less than 30 wt.-%, more preferably less than 10 wt.-%, most preferably less than 5 wt.-%, particularly less than 1 wt.-% naphthenes. Naphthenes may be precursors for forming coke but also for forming aromatics in the catalytic cracking. However, cyclic structures are less good precursors for propylene formation. Thus, for maximizing the bio-propylene productivity, lower naphthenes content in the hydrotreated bio-based hydrocarbon feeds are desired.

In certain embodiments, in step (B/B′), the hydrotreated bio-based hydrocarbon feed comprises, based on the total weight of the hydrotreated bio-based hydrocarbon feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C11 or a carbon number of at least C14. With this kind of hydrotreated bio-based hydrocarbon feed it is possible to produce a broader variety of different cracking product fractions with good productivity. Hydrotreated bio-based hydrocarbon feeds comprising the specified amounts of specified carbon numbers are obtainable e.g. by subjecting the hydrotreatment effluent and/or the hydrotreated bio-based hydrocarbon feed containing less than 1.0 wt.-% of gaseous compounds (NTP) to a fractionation. Hydrocarbon feed comprising mainly C11 or larger hydrocarbons yield a cracking product fraction rich in C5-C10 hydrocarbons usable as a component for gasoline and/or solvent compositions, in addition to the cracking product fraction(s) comprising shorter products including propylene. Additionally a fraction comprising cracked and unconverted C11 and larger hydrocarbons is obtained, which may have increased isoparaffinicity compared to the same carbon number fraction of the fresh hydrotreated bio-based hydrocarbon feed. In this way the fraction comprising the unconverted C11 and larger hydrocarbons may have higher value as a recycle feed, compared to the corresponding hydrocarbon fraction of the fresh hydrotreated bio-based hydrocarbon feed, as enhancing propylene/total C3 ratio, productivity of bio-aromatics, and productivity and quality of the bio-gasoline component in the catalytic cracking. Similarly the fraction comprising unconverted C11 and larger hydrocarbons may have higher value, preferably after a hydrotreatment such as hydrogenation of olefins, as a component for aviation and/or diesel fuel compositions having good cold properties. Furthermore, the longer saturated hydrocarbons crack at less severe conditions, compared to shorter saturated hydrocarbons, and produce a highly olefinic C5-C10 fraction that, when recovered from the cracking effluent and incorporated as a cracking effluent recycle feed to the catalytic cracking feed, again crack more easily compared to saturated C5-C10 fraction.

In certain preferred embodiments, in step (B/B′), the hydrotreated bio-based hydrocarbon feed comprises, based on the total weight of the hydrotreated bio-based hydrocarbon feed: isoparaffins and n-paraffins and the sum of the wt.-% amounts of isoparaffins and n-paraffins in the hydrotreated bio-based hydrocarbon feed is at least more than 80 wt.-%, preferably more than 90 wt.-% or even more than 95 wt.-%; more than 80 wt.-%, preferably more than 90 wt.-% hydrocarbons having a carbon number of at least C11 or a carbon number of at least C14; and more than 4 wt.-%, such as more than 5 wt.-%, preferably more than 30 wt.-% isoparaffins. These embodiments provide the combined benefits of high paraffinicity of the feed, possibility to produce a broader variety of different cracking product fractions with good productivity, as well as the benefits of elevated isoparaffin content including enhancing propylene/total C3 ratio, productivity of bio-aromatics, and productivity and quality of the bio-gasoline component in the catalytic cracking.

In certain other preferred embodiments, in step (B/B′), the hydrotreated bio-based hydrocarbon feed comprises, based on the total weight of the hydrotreated bio-based hydrocarbon feed: isoparaffins and n-paraffins and the sum of the wt.-% amounts of isoparaffins and n-paraffins in the hydrotreated bio-based hydrocarbon feed is at least 80 wt.-%, preferably more than 90 wt.-% or even more than 95 wt.-%; more than 80 wt.-%, preferably more than 90 wt.-%, more preferably more than 95 wt.-% hydrocarbons having a carbon number in the range from C5 to C10; and more than 30 wt.-%, preferably more than 40 wt.-%, more preferably more than 50 wt.-% isoparaffins. These embodiments provide the combined benefits of high paraffinicity of the feed, as well as the benefits of elevated isoparaffin content including enhancing propylene/total C3 ratio, productivity of bio-aromatics, and productivity and quality of the bio-gasoline component in the catalytic cracking. Furthermore with this kind of lighter feed higher propylene yields are obtainable especially in a once-through process (i.e. without using recycle feed), that would produce quite a lot C5+ products if using C10+ hydrocarbons (hydrocarbons having more than 10 carbon atoms) as feed. In turn C5-C10 hydrocarbons are expected to crack with better yield to propylene when the conditions, especially temperature and catalyst, are suitably selected.

The hydrotreated bio-based hydrocarbon feed may have a biogenic carbon content of more than 50 wt.-%, especially more than 60 wt.-% or more than 70 wt.-%, preferably more than 80 wt.-%, more preferably more than 90 wt.-% or more than wt.-%, even more preferably about 100 wt.-%, based on the total weight of carbon in the hydrotreated bio-based hydrocarbon feed (EN 16640 (2017)). When no fossil-based co-feed is used in the catalytic cracking feed, the cracking effluent has essentially the same biogenic carbon content as the hydrotreated bio-based hydrocarbon feed. When a co-feed is used in the catalytic cracking feed, the co-feed may have a biogenic carbon content of less than 50 wt.-%, especially less than 40 wt.-% or less than 30 wt.-%, preferably less than 20 wt.-%, more preferably less than 10 wt.-% or less than 5 wt.-%, even more preferably about 0 wt.-%, based on the total weight of carbon in the co-feed (EN 16640 (2017)). The cracking effluent, the bio-propylene composition and/or the bio-gasoline component may have a biogenic carbon content of more than 50 wt.-%, especially more than 60 wt.-% or more than 70 wt.-%, preferably more than 80 wt.-%, more preferably more than 90 wt.-% or more than 95 wt.-%, even more preferably about 100 wt.-%, based on the total weight of carbon in the hydrotreated bio-based hydrocarbon feed (EN 16640 (2017)).

In certain embodiments the hydrotreated bio-based hydrocarbon feed contains, based on the total weight of the hydrotreated bio-based hydrocarbon feed, at most 3 wt.-%, preferably at most 1 wt.-%, more preferably at most 0.5 wt.-% oxygen calculated as elemental 0.

In certain embodiments the hydrotreated bio-based hydrocarbon feed contains, based on the total weight of the hydrotreated bio-based hydrocarbon feed, at most 60 wt.-ppm, preferably at most 40 wt.-ppm, at most 20 wt.-ppm, at most 10 wt.-ppm, at most 5 wt.-ppm, at most 2 wt.-ppm or at most 1 wt.-ppm nitrogen calculated as elemental N.

In certain embodiments the hydrotreated bio-based hydrocarbon feed contains, based on the total weight of the hydrotreated bio-based hydrocarbon feed, at most 60 wt.-ppm, preferably at most 10 wt.-ppm, at most 8 wt.-ppm, at most 6 wt.-ppm, at most 4 wt.-ppm, at most 2 wt.-ppm or at most 1 wt.-ppm sulphur calculated as elemental S.

The content of nitrogen (N) content may be determined in accordance with ASTM-D4629. Contents of sulphur and oxygen may be determined using known methods, e.g. S (ASTM-D6667) and O (ASTM-D5622). Contents of carbon (C), hydrogen (H) and others may be determined by elemental analysis using e.g. ASTM D5291.

Oxygen, nitrogen, sulphur, and other heteroatoms may be present in the hydrotreated bio-based hydrocarbon feed as impurities, whether in the structure of heteroatom-containing hydrocarbons or of non-hydrocarbon compounds. These compounds are, however, undesired since they may have negative impact on catalytic cracking catalyst life and/or catalytic cracking product distribution. For example sulphur and nitrogen tend to cause catalyst fouling and/or deactivation of active sites. Additionally heteroatom containing cracking products could be formed that may be difficult to remove from the desired cracking product hydrocarbons having similar distillation behaviour. Nitrogen and oxygen may also form problematic compounds in the catalytic cracking effluent, such as basic nitrogen compounds that are corrosive and light alcohols and aldehydes that follow the product streams and may even combine to form explosive gums if diolefins are also present, particularly in cooling sections. By using a gas-depleted hydrotreated bio-based hydrocarbon feed, containing less than 1.0 wt.-%, preferably less than 0.8 wt.-%, more preferably less than 0.5 wt.-%, of gaseous compounds (NTP), it is possible to contribute to and control the heteroatom content entering the cracking reactor. Hydrotreatment of an oxygen-containing bio-based feedstock may efficiently release heteroatoms from the structure of heteroatom-containing hydrocarbons, and the formed gases, such as CO, CO₂, NH₃, and/or H₂S gases, can be easily removed from the hydrotreatment effluent e.g. by conventional gas-liquid separation techniques to achieve the desired low level of gaseous compounds (NTP) in the hydrotreated bio-based hydrocarbon feed.

In certain embodiments the hydrotreated bio-based hydrocarbon feed comprises, based on the total weight of the hydrotreated bio-based hydrocarbon feed, at most wt.-%, preferably at most 3 wt.-%, more preferably at most 2 wt.-%, even more preferably at most 1 wt.-% hydrocarbons having a carbon number of at least C22. Heavy resins and particulate matter, if present, tend to cause catalyst fouling, deactivation of active sites and pore plugging. Additionally metal impurities, that tend to cause fouling of active sites and pores of the catalytic cracking catalyst, may accumulate in the higher boiling hydrocarbon fraction. For ensuring enhanced catalyst lifetime it may thus be beneficial e.g. to fractionate the hydrotreated bio-based hydrocarbon feed so that it contains only low amounts of hydrocarbons having a carbon number of at least C22.

The Catalytic Cracking Feed, an Optional Cracking Effluent Recycle Feed and an Optional Co-Feed

The catalytic cracking feed used in the process of the present disclosure comprises a hydrotreated bio-based hydrocarbon feed containing less than 1.0 wt.-% of gaseous compounds (NTP). While the specified hydrotreated bio-based hydrocarbon feed is preferably prepared by (A) hydrotreating an oxygen-containing bio-based feedstock to obtain a hydrotreatment effluent comprising oxygen-depleted hydrocarbons, and subjecting the hydrotreatment effluent to a gas-liquid separation, the present process is not limited to said preparation. Generally the catalytic cracking feed may comprise a hydrotreated bio-based hydrocarbon feed prepared by any method as long as it contains less than 1.0 wt.-% of gaseous compounds (NTP). Preferably the catalytic cracking feed contains less than 1.0 wt.-% of gaseous compounds (NTP).

In certain embodiments the wt.-% amount of the hydrotreated bio-based hydrocarbon feed in the catalytic cracking feed is more than 80 wt.-%, such as more than 90 wt.-%, preferably more than 95 wt.-%, more preferably at least 99 wt.-%, based on the total weight of the catalytic cracking feed. By incorporating a high amount of the hydrotreated bio-based hydrocarbon feed in the catalytic cracking feed, the biogenic carbon content of also the catalytic cracking products can be increased. As the hydrotreated bio-based hydrocarbon feed has relatively low content of impurities/contaminants, due to the purifying effect of the hydrotreatment, and gas-depletion of the hydrotreatment effluent, incorporating a high amount of the hydrotreated bio-based hydrocarbon feed in the catalytic cracking feed enhances cracking catalyst performance and lifetime, contributing to cracking product yields and distribution.

In certain embodiments the catalytic cracking feed further comprises a cracking effluent recycle feed. In certain embodiments the wt.-% amount of the cracking effluent recycle feed in the catalytic cracking feed is more than 10 wt.-% or more than 20 wt.-% or more than 30 wt.-% or more than 40 wt.-% or more than 50 wt.-% or more than 60 wt.-% or more than 70 wt.-% or more than 80 wt.-% or more than 90 wt.-%, and less than 99 wt.-% or less than 90 wt.-% or preferably less than 80 wt.-% or less than 70 wt.-% or less than 60 wt.-% or less than 50 wt.-% or less than 40 wt.-% or less than 30 wt.-% or less than 20 wt.-%, based on the total weight of the catalytic cracking feed, preferably more than 10 wt.-% to less than 80 wt.-%. In the present disclosure the cracking effluent recycle feed means a portion of the catalytic cracking effluent that is recycled back to the catalytic cracking reactor.

Incorporating a cracking effluent recycle feed to the catalytic cracking feed may provide several benefits. First of all it enhances productivity of the cracking products as allowing unconverted feed components, i.e. components that were not split in the catalytic cracking into compounds having a smaller carbon number, recycled to the catalytic cracking feed to crack during the subsequent cracking cycle(s). The amount of unconverted feed may vary e.g. depending on the used process conditions, so the amount and composition of the available cracking effluent recycle feed may also vary. Although the carbon number of unconverted hydrocarbons remain unchanged during catalytic cracking, a significant portion may have reacted chemically. For example, unconverted hydrocarbons may in the catalytic cracking react into isoparaffins. Accordingly, the cracking effluent recycle feed may have a high isoparaffin content. In certain embodiments wherein the catalytic cracking feed comprises a cracking effluent recycle feed, the wt.-% amount of isoparaffins in the cracking effluent recycle feed may be at least the same as the wt.-% amount of isoparaffins in the hydrotreated bio-based hydrocarbon feed, or even higher. The wt.-% amount of isoparaffins in the cracking effluent recycle feed is calculated based on the total weight of the cracking effluent recycle feed, and the wt.-% amount of isoparaffins in the hydrotreated bio-based hydrocarbon feed is calculated based on the total weight of the hydrotreated bio-based hydrocarbon feed. In embodiments wherein the wt.-% amount of isoparaffins in the cracking effluent recycle feed is at least the same as that of the hydrotreated bio-based hydrocarbon feed, the cracking effluent recycle feed does not reduce, and advantageously even increases, the isoparaffin content of the catalytic cracking feed, thereby enhancing propylene/total C3 ratio, productivity of bio-aromatics, and productivity and quality of the gasoline component, in the catalytic cracking. Similarly, the unconverted hydrocarbons, although not cracked, may have elevated content of olefins that, when recycled as a cracking effluent recycle feed to the catalytic cracking feed, crack more easily compared to the corresponding saturated hydrocarbons. Furthermore, the cracking effluent contains elevated amounts of naphthenes and olefins, whereof naphthenes are more susceptible to converting into aromatics and/or isoparaffins and olefins are more susceptible to cracking into shorter hydrocarbons, compared to paraffins, recycling at least a portion of the cracking effluent as a cracking effluent recycle feed to the catalytic cracking feed may provide improved productivity of bio-aromatics and other cracking products. Recycling is particularly suitable when the catalytic cracking feed has a low impurity content, as then the recycling does not cause accumulation of catalyst poisons and/or coke-forming compounds in the reactor in a harmful extent. In this way the catalyst life-time and/or regeneration period may be enhanced. The catalytic cracking feed has a reduced impurity content e.g. when it comprises only low or no amount of a co-feed containing elevated amounts of impurities/contaminants. On the other hand, coke-formation on the catalyst may be desired to certain extent, so as to improve the overall energy-efficiency of the present process, so recycling may help with the energy-efficiency. This is because the cracking effluent contains higher amount of coke-forming compounds, especially aromatics, naphthenes and olefins, compared to the fresh hydrotreated bio-based hydrocarbon feed, so recycling at least a portion thereof is expected to enhance coke-formation on the catalyst and thus energy released during catalyst regeneration. By selecting a suitable fraction of the cracking effluent for recycling and/or by adjusting the amount of the cracking effluent recycle feed in the catalytic cracking feed, it is possible to increase the coke-formation in a controlled manner. Depending on which benefits are desired to be emphasized, the amount of the cracking effluent recycle feed in the catalytic cracking feed may vary.

In certain embodiments the sum of the wt.-% amounts of the hydrotreated bio-based hydrocarbon feed and the cracking effluent recycle feed in the catalytic cracking feed is more than 80 wt.-%, such as more than 85 wt.-% or more than 90 wt.-%, preferably more than 95 wt.-% such as more than 97 wt.-%, more preferably at least 99 wt.-%, based on the total weight of the catalytic cracking feed. These embodiments provide the combined benefits of incorporating a high amount of the hydrotreated bio-based hydrocarbon feed in the catalytic cracking feed, and incorporating a cracking effluent recycle feed to the catalytic cracking feed, as discussed above.

In certain embodiments the weight ratio of the hydrotreated bio-based hydrocarbon feed and the cracking effluent recycle feed in the catalytic cracking feed is at least 10:90, preferably at least 20:80, more preferably at least 50:50, such as at least 80:20, and/or at most 99:1, such as at most 90:10, preferably at most 80:20, such as at most 50:50, or at most 20:80. By suitably selecting the weight ratio of the hydrotreated bio-based hydrocarbon feed and the cracking effluent recycle feed in the catalytic cracking feed, it is possible to emphasize the benefits of incorporating a high amount of the hydrotreated bio-based hydrocarbon feed in the catalytic cracking feed, or the benefits of incorporating a cracking effluent recycle feed to the catalytic cracking feed, as discussed above, while still achieving combined benefits of both, to certain extent.

In certain embodiments the process further comprises recovering from the cracking effluent a fraction of hydrocarbons having a carbon number of at least C5, and incorporating at least a portion of said fraction as a cracking effluent recycle feed to the catalytic cracking feed.

Recovering from the cracking effluent a fraction of hydrocarbons having a carbon number of at least C5, and incorporating at least a portion of said fraction as a cracking effluent recycle feed to the catalytic cracking feed, is beneficial as it is possible to produce a broad variety of different cracking products with good conversion-normalized yields. Hydrocarbon feed comprising mainly C11 or larger hydrocarbons yield a cracking product fraction rich in C5-C10 hydrocarbons usable as a component for gasoline and/or solvent compositions, in addition to the cracking product fraction(s) comprising shorter products including propylene. Additionally a fraction comprising cracked and unconverted C11 and larger hydrocarbons is obtained, which may have increased isoparaffin content compared to that of the fresh hydrocarbon feed, so that also this fraction comprising the unconverted C11 and larger hydrocarbons may have higher value as a recycle feed, compared to the fresh hydrotreated bio-based hydrocarbon feed, as enhancing propylene/total C3 ratio, productivity of aromatics, and productivity and quality of the gasoline component, in the catalytic cracking. Additionally the fraction comprising cracked and unconverted C11 and larger hydrocarbons, potentially having increased isoparaffin content compared to that of the fresh hydrotreated bio-based hydrocarbon feed, may have higher value, preferably after a hydrotreatment such as hydrogenation of olefins, as a component for aviation and/or diesel fuel compositions having good cold properties, compared to the fresh hydrotreated bio-based hydrocarbon feed. Furthermore, the longer saturated hydrocarbons crack at less severe conditions, compared to shorter saturated hydrocarbons, and produce a highly olefinic C5-C10 fraction that, when recovered from the cracking effluent and incorporated as a cracking effluent recycle feed to the catalytic cracking feed, again crack more easily compared to saturated C5-C10 fraction.

In certain embodiments the process further comprises recovering at least bio-aromatics from the fraction of hydrocarbons having a carbon number of at least C5 before incorporating at least a portion of said fraction as a cracking effluent recycle feed to the catalytic cracking feed. In this way a further valuable product, bio-aromatics, is recovered from the cracking effluent, instead of consuming it for coke-formation on the cracking catalyst. Aromatics are large volume commodity chemicals with diverse applications such as (from benzene) ethyl benzene, cumene, cyclohexane, nitrobenzene, (from toluene) toluene diisocyanate, benzoic acid (from para-xylene) terephthalic acid for PET and (from ortho-xylene) phthalic anhydride (plasticiser in PVC). As with propylene, bio-based aromatics are not trivial to fabricate.

In certain embodiments the process further comprises hydrotreating, such as hydrogenating, the fraction of hydrocarbons having a carbon number of at least C5, or the cracking effluent recycle feed, before incorporating to the catalytic cracking feed. In this way it is possible to reduce or remove e.g. diolefins that are capable of forming explosive gums with other compounds potentially present in the cracking effluent.

In certain embodiments the cracking effluent recycle feed comprises, based on the total weight of the cracking effluent recycle feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C5, or a carbon number of at least C11 or a carbon number of at least C14.

In certain embodiments the cracking effluent recycle feed and the hydrotreated bio-based hydrocarbon feed comprise, based on the total weight of the cracking effluent recycle feed or the hydrotreated bio-based hydrocarbon feed, more than 50 wt.-%, preferably more than 60 wt.-%, further preferably more than 70 wt.-%, more preferably more than 80 wt.-%, and even more preferably more than 90 wt.-% hydrocarbons having a carbon number of at least C5, or a carbon number of at least C11 or a carbon number of at least C14. The more similar the carbon chain lengths of the hydrotreated bio-based hydrocarbon feed and the cracking effluent recycle feed are, the easier it is to optimize the cracking conditions in a single reactor for producing the desired cracking products. Also better blendability with each other may be expected, so that the catalytic cracking feed comprising the hydrotreated bio-based hydrocarbon feed and the cracking effluent recycle feed less likely forms two or multiple phase systems even in the absence of sufficient mixing, thereby reducing variation in the composition of the cracking effluent fractions.

In certain embodiments the catalytic cracking feed further comprises, based on the total weight of the catalytic cracking feed, less than 50 wt.-%, preferably less than 20 wt.-%, more preferably less than 10 wt.-%, or less than 5 wt.-% a co-feed selected from a fossil-based co-feed, a fatty co-feed, a co-feed of thermally and/or enzymatically liquefied biomass, and any combinations thereof. As the hydrotreated bio-based hydrocarbon feed has a low impurity content, it is possible to incorporate in the catalytic cracking feed also some amounts of a co-feed containing elevated amounts of impurities or contaminants, without essentially harming the catalytic cracking process. This provides desired flexibility to the running of the process as there may be limitations in the availability of the hydrotreated bio-based hydrocarbon feed, necessitating use of a co-feed.

In preferred embodiments, the process of the present disclosure involves coke deposition on the solid catalyst and regeneration thereof by burning the coke, and utilising the generated thermal energy further in the catalytic cracking reactor. As the hydrotreated bio-based hydrocarbon feed itself is a valuable resource, it may be desired to incorporate in the catalytic cracking feed some amounts of a less valuable co-feed, containing compounds that have higher selectivity to coke-formation, compared to the typical compounds present in the hydrotreated bio-based hydrocarbon feed. Examples of compounds having higher selectivity to coke-formation include naphthenes, aromatics, olefins, and/or heteroatom-containing hydrocarbons, typically organic oxygenates, such as alcohols, aldehydes, ketones, carboxylic acids, ethers, esters, and anhydrides; organosulphur compounds, such as thiols, organic sulphides and disulphides, and thiophenes; or organonitrogen compounds, such as amines, diamines, amides, pyrroles, piperidines, quinolines and pyridines. Examples of co-feeds that comprise elevated amounts of these compounds include e.g. fossil-based co-feeds, fatty co-feeds, and co-feeds of thermally and/or enzymatically liquefied biomass. For example, the co-feed may comprise more than 20 wt.-% or more than 30 wt.-% or more than 40 wt.-% or more than 50 wt.-%, based on the total weight of the co-feed, one or more of naphthenes, aromatics, olefins, organic oxygenates, organosulphur compounds and organonitrogen compounds, calculated as the total amount of naphthenes, aromatics, olefins and elemental O, S and N in the co-feed. Incorporating minor or no amounts of a co-feed comprising heteroatom-containing hydrocarbons to the catalytic cracking feed may be beneficial so as to ensure efficient cleavage of the heteroatoms covalently bound to the hydrocarbons, under the used catalytic cracking conditions. In case the cleavage of the heteroatoms from the hydrocarbon structure is compromised, smaller hydrocarbon moieties, such as shorter alcohols, thiols etc, formed by cracking the heteroatom-containing hydrocarbons contained in the co-feed would end-up in the cracking effluent. Depending on the desired end-use, and the specifications that the cracking effluent fractions would need to meet, cumbersome purification steps of the cracking effluent might be required.

In certain embodiments the catalytic cracking feed comprises, based on the total weight of the catalytic cracking feed, at least 0.5 wt.-%, preferably at least 1.0 wt.-%, at least 3.0 wt.-%, at least 5.0 wt.-%, or at least 10.0 wt.-% (total) aromatics. As aromatics are coke-precursors, these embodiments may enhance energy-efficiency of the present process.

In certain embodiments the catalytic cracking feed has a biogenic carbon content of more than 50 wt.-%, or more than 60 wt.-%, preferably more than 70 wt.-%, such as more than 80 wt.-% or more than 90 wt.-%, more preferably more than 95 wt.-%, based on the total weight of carbon in the catalytic cracking feed (EN 16640 (2017)). In this way a high biogenic carbon content is obtainable also to the cracking products.

In addition to the cracking effluent recycle feed and/or the co-feed, a coking precursor additive may be incorporated to the catalytic cracking feed. If insufficient or no amount of cracking effluent recycle feed, co-feed or coking additive is incorporated to the catalytic cracking feed, from time to time or at all, insufficient coking is expected to occur, necessitating use of external power/fuel for heating the catalyst. On the other hand, low coking tendency means that most of the hydrotreated bio-based hydrocarbon feed is upgraded to valuable products rather than being lost as coke. Therefore, external heating of the catalyst in a regeneration step may be more favourable than addition of a coking additive and/or a co-feed, and/or using a high recycling ratio.

The Catalytic Cracking

In the process of the present disclosure the catalytic cracking feed is subjected to catalytic cracking in a catalytic cracking reactor at a temperature of at least 450° C. using a moving solid catalyst, to obtain a cracking effluent.

Catalytic cracking processes using moving solid catalyst, especially fluid catalytic cracking process, are based on catalytic cracking reactions. They are distinct from other industrial processes involving cracking of hydrocarbons: e.g. steam cracking is based on thermal cracking reactions, generating light olefins with huge energy consumption; hydrocracking is based on catalytic cracking reactions in the presence of a catalyst and added molecular hydrogen, generating saturated hydrocarbons; catalytic reforming is based on dehydrogenation, isomerization, aromatization and hydrocracking reactions in the presence of a catalyst and a high partial pressure of added molecular hydrogen (typically 5-45 atm), converting n-paraffins into isoparaffins and cyclic naphthenes, that are further dehydrogenated to high-octane aromatic hydrocarbons, also generating significant amounts of hydrogen gas as a by-product.

The catalytic cracking process of the present disclosure provides several advantages over steam cracking. The present process has far higher energy efficiency compared to steam cracking, which is the current industry standard for propylene manufacturing. The present process also involves less formation of ethylene and CH₄, and provides surprisingly high propylene (C3=) productivity compared to steam cracking. In addition, a far higher propylene to ethylene (C3=/C2=) weight ratio can be achieved by the present process. This is especially beneficial as using currently available technologies it is more difficult to produce propylene from bio-based raw materials, than ethylene. Furthermore, unlike for steam cracking, in the present process there is no need to add sulphur to the cracking feed, so there is less requirement for gas washing, and easier purification of the cracking product.

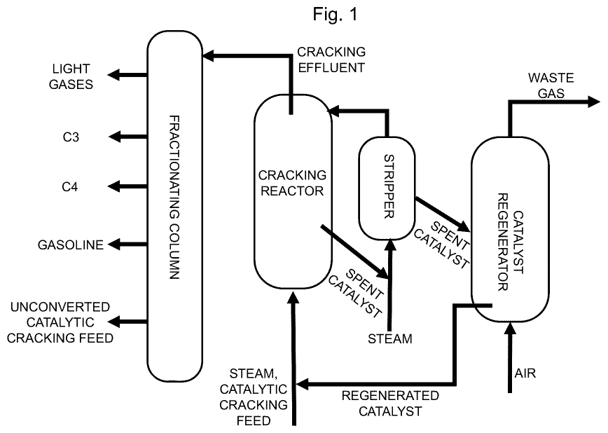

The catalytic cracking process using a moving solid catalyst allows an excellent integration of the cracking reactor and catalyst regenerator that provides the highest thermal efficiency, as can be seen e.g. from FIG. 1 showing a schematic drawing of a process according to an example embodiment. In the embodiment of FIG. 1, a fluidized-bed (or fluid-bed) of catalyst particles is brought into contact with the catalytic cracking feed along with a carrier gas, e.g. injected steam, at the entrance (called the riser) of the reactor. The hot catalyst particles coming from the regenerator unit evaporate the hydrocarbons in the catalytic cracking feed upon contact in the riser, and the cracking starts as the hydrocarbon vapours and the catalyst particles move upward in the reactor. The temperature of the catalyst particles drops as the evaporation of the catalytic cracking feed and endothermic cracking reactions proceed during the upward movement. Cracking reactions also deposit coke on the catalysts, leading to the deactivation of the catalyst. After removing the adsorbed hydrocarbons e.g. by steam stripping, the coked catalyst is sent to the regeneration unit to burn off the coke with air. Heat released from burning the coke deposit increases the temperature of the catalyst particles that are returned to the riser to complete the cycle. Burning off the coke in the regenerator provides the energy necessary for cracking without much loss, thus increasing the thermal efficiency of the process. The cracking products are sent to the fractionator for recovery after they are separated from the catalyst particles in the upper section of the reactor. Since the catalytic cracking process using a moving solid catalyst is a continuous process, there is no need to take a reactor offline for regenerating the catalyst. This is different from processes using fixed bed reactors that must be taken off-line to burn off the coke, or regenerate the catalyst, which means lost productivity.

In the catalytic cracking reactor, the cracking reactions initiate on the active sites of the solid catalysts with the formation of carbocations, and the subsequent ionic chain reactions produce inter alia light olefins, isoparaffins and aromatics to constitute the cracking product stream that is sent e.g. to a fractionator for recovering at least a fraction rich in bio-propylene as the bio-propylene composition, and optionally also: a fraction rich in C5-C10 hydrocarbons as the bio-gasoline component, a fraction rich in bio-aromatics, and a fraction comprising unconverted catalytic cracking feed. A carbon-rich by-product of catalytic cracking, termed “coke,” deposits on catalyst surfaces and blocks the active sites. The coke deposited on the catalyst surface and eventually burned off for heat is rich in carbon and thus enables the production of large quantities of light cracking products. Using the present process it is possible to produce bio-propylene composition with significantly lower energy consumption compared to steam cracking.

Different configurations of the commercial catalytic cracking processes using moving solid catalyst, especially FCC processes, exist with different positions of the reactor and the regenerator: they can be side by side or stacked, where the reactor is mounted on top of the regenerator. Major licensor companies that offer FCC processes with different configurations include Kellogg Brown & Root, CB&I Lummus, ExxonMobil Research and Engineering, Shell Global Solutions International, Stone & Webster Engineering Corporation, Institut Francais du Petrole (IFP), and UOP. The UOP design of high-efficiency two-stage regenerator units offer advantages of uniform coke burn, higher conversion of CO to CO₂ and lower NOx emissions among others. Further modifications to FCC plants include an installation of a catalyst cooler, which may provide better control of the catalyst/oil ratio; the ability to optimize the FCC operating conditions, increase conversions, and process heavier catalytic cracking feeds; and better catalyst activity and catalyst maintenance. Any of the commercial catalytic cracking configurations could be used in the process of the present disclosure.

Examples of suitable reactors for performing the catalytic cracking process of the present disclosure include transported bed reactors and fluidized bed reactors. Most preferably the catalytic cracking reactor comprises a riser. Within the reactor, the catalytic cracking feed can be contacted with a moving solid catalyst under cracking conditions thereby resulting in spent catalyst particles containing carbon deposited thereon and a lower boiling catalytic cracking effluent.

In preferred embodiments, step (C) comprises catalytically cracking the catalytic cracking feed in a fluid catalytic cracking reactor, preferably a fluid catalytic cracking reactor comprising a riser, at a temperature of at least 450° C. using a fluidized solid catalyst to obtain a cracking effluent. Processes according to these embodiments may be referred to as fluid catalytic cracking (FCC) processes. Particles of the solid catalyst may be fluidized for example by vaporized catalytic cracking feed, steam and/or air. Preferably at least vaporized catalytic cracking feed and steam are used for fluidizing the solid catalyst particles.

Other gases may be present in the reactor, especially gases which are produced in the course of the catalytic cracking reactions such as hydrogen. However preferably no molecular hydrogen is added to the catalytic cracking reactor, because the process of the present disclosure is a process for manufacturing propylene, i.e. an unsaturated compound. Preferably, the catalytic cracking feed and all of its constituents i.e. the hydrotreated bio-based hydrocarbon feed, the optional cracking effluent recycle feed, and the optional co-feed, are essentially free from molecular hydrogen (H₂).

The cracking effluent, comprising the cracking products, can be removed from the catalyst particles using known methods and equipment. Preferably this can be done with mechanical separation devices, such as a cyclone. The cracking effluent can be removed from the reactor via an overhead line, cooled and sent to e.g. a fractionator tower for recovering of the various cracking products.

In certain embodiments step (C) further comprises separating the cracking effluent and the spent solid catalyst, regenerating the spent solid catalyst outside the catalytic cracking reactor and re-introducing at least part of the regenerated solid catalyst into the cracking reactor.

In certain embodiments regenerating the solid catalyst comprises burning coke formed on the catalyst to regenerate and heat the catalyst, and optionally further heating the catalyst during and/or after the regeneration with an external heating source.

External heating source is a source of heat other than heat generated internally in the present process, particularly heat generated by burning coke (or other deposits, adhered or absorbed material) on the solid catalyst. External heating source may be a fuel added when burning coke, hot air (or other gas or gas composition) externally heated, indirect heating by radiation (e.g. IR) or direct heating, e.g. on a heating plate or the like.

In certain embodiments the catalytic cracking is conducted in the catalytic cracking reactor at a temperature of at least 450° C., or at least 500° C., or at least 520° C., and/or less than 700° C., or less than 680° C., or less than 650° C., or less than 600° C., or less than 580° C., or less than 550° C., preferably at least 500° C. to less than 700° C., more preferably at least 520° C. to less than 680° C.

In certain embodiments the catalytic cracking is conducted in the catalytic cracking reactor at hydrocarbon partial pressures from about 5 kPa to 500 kPa (absolute), preferably from about 5 to 300 kPa (absolute), such as from about 10 to 250 kPa (absolute).

In certain embodiments the catalytic cracking is conducted using a catalyst-to-oil-ratio of at least 1.0, preferably at least 2.0, or at least 4.0; and/or at most 30, preferably at most 20, or at most 15.

In certain embodiments in step (C) the contact time of the catalytic cracking feed with the solid catalyst is at most 10 seconds, preferably at most 8 seconds, or at most 7 seconds, or at most 6 seconds, or at most 5 seconds, or at most 4 seconds, or at most 3 seconds. Typically the contact time of the catalytic cracking feed with the solid catalyst in the present invention is from about 2 seconds to about 5 seconds. Short contact times are beneficial to avoid or at least reduce the risk that the bio-propylene and/or potential other olefinic cracking products would start to polymerize. On the other hand too short contact times may decrease the cracking reactions and thus reduce cracking product yields.

In general, any commonly known particulate catalytic cracking catalyst may be employed in the process of the present disclosure. In particular, the catalyst may include any of the catalysts that are used in the art of FCC.

Typical FCC catalysts usable in the present invention consist of a fine powder with an average particle size of 60-75 μm and a size distribution ranging from 20 to 120 μm.

Typically at least zeolite-type material is present in the catalyst. Other typical components that may additionally be present in the catalysts include active matrix, filler, and binder. Of these components the zeolite-type material is more active and may provide selectivity for specific cracking products.

A single catalyst may be used alone or a combination of two or more catalysts may be used.

Typically the solid catalyst is a solid acidic catalyst.

In certain embodiments the solid catalyst comprises one or more zeolite-type materials.

In certain embodiments the solid catalyst comprises one or more zeolite-type materials selected from large-pore zeolites, such as Y-zeolite, and medium-pore zeolites, such as ZSM-5 or ZSM-23. Preferably the solid catalyst comprises at least ZSM-5.

In certain embodiments the solid catalyst comprises a combination of two or more zeolite-type materials selected from large-pore zeolites, such as Y-zeolite, and medium-pore zeolites, such as ZSM-5 or ZSM-23.

In certain embodiments the solid catalyst may comprise one or more zeolite-type materials selected from small-pore zeolites, such as SAPO-34. In certain embodiments the solid catalyst comprises a combination of one or more zeolite-type materials selected from small-pore zeolites, such as SAPO-34, and one or more zeolite-type materials selected from large-pore zeolites, such as Y-zeolite, and medium-pore zeolites, such as ZSM-5 or ZSM-23.

In certain embodiments the solid catalyst comprises a zeolite-type material doped with one or more metals, e.g. with a transition metal and/or a lanthanide. The doping may be for example impregnation (with solution of the metal/ion, followed by drying) or ion exchange reaction.

In certain embodiments the solid catalyst comprises an inert filler, such as kaolin.

In certain embodiments the solid catalyst comprises a binder, such as silica or alumina.

In certain embodiments the solid catalyst comprises an active matrix, such as alumina material.

In certain embodiments the solid catalyst comprises one or more zeolite-type materials selected from large-pore zeolites, such as Y-zeolite, and medium-pore zeolites, such as ZSM-5 or ZSM-23; a binder, such as silica or alumina; an inert filler, such as kaolin; and an active matrix, such as alumina material.

Large-pore-size zeolites that can be used in the catalysts of the present process include those having pores with average pore diameter greater than 0.7 nm, and typically having 12 membered rings. Pore Size Indices of large pores are preferably above 31. Usable large-pore-size zeolites include both natural and synthetic large-pore-size zeolites. Non-limiting examples of usable natural large-pore zeolites include gmelinite, faujasite, offretite, and mordenite. Suitable large-pore zeolites for use herein include particularly zeolite Y, USY (ultra stable Y), and REY (rare earth Y).

Medium-pore-size zeolites that can be used in the catalysts of the present process include those described in “Atlas of Zeolite Structure Types,” eds. W. H. Meier and D. H. Olson, Butterworth-Heineman, Third Edition, 1992, which is hereby incorporated by reference. The medium-pore-size zeolites generally have a pore size from 0.5 nm to 0.7 nm and include for example, MFI, MFS, MEL, MTW, EUO, MTT, HEU, FER, and TON structure type zeolites (IUPAC Commission of Zeolite Nomenclature). Preferred medium-pore-size zeolites include ZSM-5 and ZSM-23, most preferred being ZSM-5. Usable ZSM-5 zeolites are described e.g. in U.S. Pat. Nos. 3,702,886 and 3,770,614, and usable ZSM-23 e.g. in U.S. Pat. No. 4,076,842.

The Cracking Effluent

The cracking effluent relates to the effluent obtained directly after the catalytic cracking reactions, i.e. including liquid and gaseous products, but excluding solids, especially the spent solid catalyst.

The below embodiments relate to benefits obtainable even for once-through processes, i.e. processes where cracking effluent is not recycled back to the reactor. When using cracking effluent recycle feed, even better weight ratios and yields may be obtained.

In certain embodiments the weight ratio of propylene to ethylene in the cracking effluent is more than 1.0, such as at least 1.5, preferably more than 2.0, more preferably more than 2.5, or more than 3.0. Usually, the ratio will be 10 or less, such as 5 or less.

In certain embodiments the weight ratio of propylene to total-C3 (100%×propylene/{summed amount of propylene and propane}) in the cracking effluent is at least 65 wt.-%, such as at least 70 wt.-%, or at least 80 wt.-%, preferably at least 85 wt.-%, more preferably at least 90 wt.-%. Usually, without further purification, the weight ratio may be 97 wt.-% or less, such as 95 wt.-% or less.

In certain embodiments the conversion normalized yield of bio-propylene (100%×{weight of the bio-propylene in the cracking effluent/weight of converted catalytic cracking feed}) is more than 20 wt.-%, such as more than 22 wt.-%, preferably more than 25 wt.-%, more preferably more than 30 wt.-%.

In the context of the present disclosure weight of assumed converted catalytic cracking feed is used instead of the weight of actually converted catalytic cracking feed. The weight of the assumed converted catalytic cracking feed may be obtained e.g. by deducting weight of assumed unconverted catalytic cracking feed from the weight of the catalytic cracking feed fed to the reactor (excluding any recycle feed, if used). As the weight of the actually unconverted catalytic cracking feed would be cumbersome to determine, weight of an assumed unconverted feed is used instead. The sum of weight amounts of a cracking effluent fraction having a boiling range (initial boiling point IBP to final boiling point FBP) of 221-338° C. (standard light cycle oil (LCO)) and a cracking effluent fraction having IBP starting from 338° C. (standard heavy cycle oil (HCO)) is regarded as the weight of an assumed unconverted feed. Thus a cracking effluent fraction having FBP up to 221° C. can be regarded as the weight of the assumed converted catalytic cracking feed. Distillation characteristics may be determined by ENIS03405.

In certain embodiments the conversion normalized yield of aromatics (also referred to as “bio-aromatics”) (100%×{weight of the aromatics in the cracking effluent/weight of converted catalytic cracking feed}) is more than 1.0 wt.-%, such as more than 2.0 wt.-%, preferably more than 3.0 wt.-%, more preferably more than 5.0 wt.-%.

Recovering

A fraction rich in bio-propylene or enriched in bio-propylene, means in the context of the present disclosure that the wt.-% amount of the bio-propylene in the fraction, based on the total weight of the fraction, is higher than the wt.-% amount of the bio-propylene in the cracking effluent, based on the total weight of the cracking effluent. Preferably the wt.-% amount of the bio-propylene is higher than the wt.-% amount of any other single compound present in the fraction rich in bio-propylene, in other words that the fraction rich in bio-propylene comprises bio-propylene as the most abundant compound. More preferably the fraction rich in bio-propylene comprises more than 50 wt.-% bio-propylene, based on the total weight of the fraction rich in bio-propylene.

A fraction rich in bio-aromatics or enriched in bio-aromatics, means in the context of the present disclosure that the wt.-% amount of the bio-aromatics in the fraction, based on the total weight of the fraction, is higher than the wt.-% amount of the bio-aromatics in the cracking effluent, based on the total weight of the cracking effluent. Preferably the wt.-% amount of the bio-aromatics is higher than the wt.-% amount of any other single compound present in the fraction rich in bio-aromatics, in other words that the fraction rich in bio-aromatics comprises bio-aromatics as the most abundant compounds. More preferably the fraction rich in bio-aromatics comprises more than 50 wt.-% bio-aromatics, based on the total weight of the fraction rich in bio-aromatics.

A fraction rich in C5-C10 hydrocarbons or enriched in C5-C10 hydrocarbons, means in the context of the present disclosure that the sum of the wt.-% amounts of the C5-C10 hydrocarbons in the fraction, based on the total weight of the fraction, is higher than the sum of the wt.-% amounts of the C5-C10 hydrocarbons in the cracking effluent, based on the total weight of the cracking effluent. Preferably in the fraction rich in C5-C10 hydrocarbons, the sum of the wt.-% amount of the C5-C10 hydrocarbons is higher than the sum of the wt.-% amounts of other compounds present in the fraction rich in C5-C10 hydrocarbons, based on the total weight of the fraction rich in C5-C10 hydrocarbons.

In certain embodiments the process comprises recovering from the cracking effluent a fraction rich in bio-propylene as the bio-propylene composition, and a fraction rich in C5-C10 hydrocarbons as the bio-gasoline component. In this way a further valuable product, bio-gasoline, is recovered from the cracking effluent.

In certain embodiments the process comprises recovering from the cracking effluent a fraction rich in bio-propylene as the bio-propylene composition, and a fraction rich in bio-aromatics. In this way a further valuable product, bio-aromatics, is recovered from the cracking effluent, instead of consuming it for coke-formation on the cracking catalyst. Aromatics are large volume commodity chemicals with diverse applications such as (from benzene) ethyl benzene, cumene, cyclohexane, nitrobenzene, (from toluene) toluene diisocyanate, benzoic acid (from para-xylene) terephthalic acid for PET and (from ortho-xylene) phthalic anhydride (plasticiser in PVC). As with propylene, bio-based aromatics are not trivial to fabricate.

In certain embodiments the process comprises recovering from the cracking effluent a fraction rich in bio-propylene as the bio-propylene composition, a fraction rich in C5-C10 hydrocarbons as the bio-gasoline component, and a fraction rich in bio-aromatics. In this way two further valuable products, bio-gasoline and bio-aromatics, are recovered from the cracking effluent.

In certain embodiments recovering, especially in step (D), comprises one or more of distilling, fractionating, separating, evaporating, flash-separating, membrane separating, extracting, using extractive-distillation, using chromatography, using molecular sieve adsorbents, using thermal diffusion, complex forming, crystallizing, preferably at least fractionating, distilling, extracting, using extractive-distillation.

In certain embodiments (D) recovering from the cracking effluent a fraction rich in C5-C10 hydrocarbons as the bio-gasoline component and recovering from the cracking effluent a fraction of hydrocarbons having a carbon number of at least C5, refer to the same recovering step, and in certain embodiments to different recovering steps conducted consecutively or concurrently. For example recovering from the cracking effluent a first fraction of hydrocarbons having a carbon number of at least C5 may be followed by recovering from the first fraction a second fraction rich in C5-C10 hydrocarbons as the bio-gasoline component, before incorporating at least a portion of the first and/or of the second fraction as a cracking effluent recycle feed to the catalytic cracking feed. Alternatively recovering from the cracking effluent a second fraction rich in C5-C10 hydrocarbons as the bio-gasoline component and recovering from the cracking effluent a third fraction of hydrocarbons having a carbon number of at least C11 may be conducted concurrently e.g. by fractional distillation, before incorporating at least a portion of the second and/or of the third fraction as a cracking effluent recycle feed to the catalytic cracking feed.

Recovering may be conducted in several steps. For example, a first recovering step from the cracking effluent may produce a first bio-propylene composition (a first fraction rich in bio-propylene, comprising bio-propylene, bio-propane, C4 paraffins, C4 olefins, ethylene and ethane). Thereafter a second recovering step from the first bio-propylene composition may produce a second bio-propylene composition (a second fraction further enriched in bio-propylene, containing more of, or consisting essentially of, bio-propylene and bio-propane), as well as a fraction enriched in C4 hydrocarbons and a fraction enriched in C2 hydrocarbons. Similarly, a first recovering step from the cracking effluent may produce a first bio-gasoline component (a first fraction rich in C5-C10 hydrocarbons). Thereafter a second recovering step from the first bio-gasoline component may produce a fraction enriched in C5-C10 aromatics, and a second bio-gasoline component (a second fraction rich in C5-C10 hydrocarbons, depleted of C5-C10 aromatics). This kind of staged recovery of some of the desired fractions, such as of the bio-propylene composition and of the bio-gasoline composition, may be beneficial, e.g. when also other, close fractions are to be recovered as their own fractions.

Optionally, even further fractions may be recovered from the catalytic cracking effluent, especially a fraction rich in bio-ethylene as a bio-ethylene composition, preferably comprising more than 50 wt.-% of ethylene, based on the total weight of the bio-ethylene composition, and/or a fraction rich in C4 hydrocarbons (as a bio-C4 composition, preferably comprising more than 50 wt.-% of C4 hydrocarbons, based on the total weight of the bio-C4 composition), such as a fraction rich in C4 olefins (as a bio-butylene composition, preferably comprising more than 50 wt.-% of C4 olefins, based on the total weight of the bio-butylene composition).

Any of the recovered fractions may be subjected to one or more further purification and/or fractionation step. The optional purification and/or fractionation steps or treatments may be selected depending on the intended end use and/or desired degree of purity of the recovered bio-propylene composition, bio-gasoline component, bio-ethylene composition, bio-C4 composition, bio-butylene composition, bio-aromatics fraction, and/or any other recovered cracking effluent fraction.

For example, in certain embodiments the process further comprises hydrotreating, such as hydrogenating, the fraction of hydrocarbons having a carbon number of at least C5, and/or the bio-gasoline component. In this way it is possible to reduce or remove e.g. diolefins that are capable of forming explosive gums with other compounds potentially present the fraction; reduced olefin content may also allow higher bio-gasoline component shares to be incorporated in gasoline blends.

In certain embodiments the process further comprises removing benzene, or BTX, or (total) aromatics, from the fraction rich in C5-C10 hydrocarbons, preferably to a level of at most 1 wt.-%, based on the total weight of the fraction rich in C5-C10 hydrocarbons. This may be achieved e.g. by hydrodearomatization, solvent extraction, or any other known method. These embodiments are especially beneficial for use in gasoline compositions having an upper limit of 1 wt.-% for benzene. Low aromatics and/or benzene content may be desired in many other applications as well, such as in many household applications.

In certain embodiments the process further comprises selectively hydrotreating the fraction rich in bio-propylene to remove certain contaminants such as MAPD (propyne-propadiene mixture) and/or the fraction rich in bio-ethylene to remove certain contaminants such as acetylene. These compounds are very harmful for the quality and further use of the bio-ethylene and bio-propylene compositions.

In certain embodiments the process further comprises purifying the bio-propylene composition until the total content of the bio-propylene in the bio-propylene composition reaches at least 85 wt.-%, preferably at least 90 wt.-%, more preferably at least 95 wt.-%, even more preferably at least 99 wt.-% or at least 99.5 wt.-%, based on the total weight of the bio-propylene composition.

The Bio-Propylene Composition, Polymerization Method and Obtainable (Co)Polymer Composition

A bio-propylene composition according to the present disclosure comprises bio-propylene and bio-propane, wherein the total content of the bio-propylene is at least 80 wt.-%, based on the total weight of the bio-propylene composition, and the weight ratio of bio-propylene to bio-propane is at least 4.5; preferably the total content of the bio-propylene is at least 85 wt.-%, based on the total weight of the bio-propylene composition, and the weight ratio of bio-propylene to bio-propane is at least 5.3; more preferably the total content of the bio-propylene is at least 90 wt.-%, such as at least 99 wt.-%, based on the total weight of the bio-propylene composition, and the weight ratio of bio-propylene to bio-propane is at least 9.0. By the present process it is possible to obtain bio-propylene compositions with exceptionally high bio-propylene total content, and very low bio-propane content, providing high weight ratio of bio-propylene to bio-propane. These compositions are directly usable instead of or in addition to conventional fossil-based propylene compositions, as easily meeting or exceeding a typical refinery grade purity requirement (50-70%), or even a typical chemical grade purity requirement (90-95%), or even a typical polymer grade purity requirement (99.5% or more).

In certain embodiments the bio-propylene composition is obtainable by the process of the present disclosure.

In certain embodiments the process of the present disclosure further comprises purifying the recovered bio-propylene composition, and optionally derivatising at least a part of the bio-propylene molecules in the bio-propylene composition, to obtain a polymerizable composition of bio-monomers, such as olefinically unsaturated or epoxide bio-monomers. The purification may be conducted e.g. by any known purification technique such as distillation, extraction, selective hydrotreatment to remove MAPD, etc, further increasing the bio-propylene content of the bio-propylene composition and/or removing impurities/contaminants from the composition. The derivatising may be conducted e.g. by any known chemical modification technique providing bio-monomers e.g. with anionically and/or cationically charged group(s), hydrophobic group(s), or any other desired characteristic. The present disclosure further provides a polymerizable composition obtainable by the process of this embodiment and/or a monomer blend comprising said polymerizable composition.

In certain embodiments the process of the present disclosure further comprises providing a monomer blend comprising the polymerizable composition of bio-monomers, such as olefinically unsaturated or epoxide bio-monomers, and (co)polymerizing the bio-monomers in the polymerizable composition to obtain a (co)polymer composition. The present disclosure further provides a (co)polymer composition obtainable by the process of this embodiment.

A method for producing a (co)polymer composition according to the present disclosure comprises producing a bio-propylene composition according to the process of the present disclosure, optionally purifying the bio-propylene composition, and optionally derivatising at least a part of the bio-propylene molecules in the bio-propylene composition, to obtain a polymerizable composition of bio-monomers, such as olefinically unsaturated or epoxide bio-monomers, and (co)polymerizing a monomer blend comprising the polymerizable composition of bio-monomers to obtain the (co)polymer composition.

In certain embodiments the bio-monomer is an olefinically unsaturated bio-monomer selected from bio-propylene, bio-acrylic acid, bio-acrylonitrile, and bio-acrolein, or an epoxide bio-monomer selected from bio-propylene oxide.

In the context of the present disclosure, the monomers are meant to include the monomers in any form, including e.g. free, salt and ester forms, and/or carrying any side group such as a methyl, ethyl etc side group. For example the acrylic acid monomer is meant to include e.g. (meth)acrylic acid, (meth)acrylic acid esters, (meth)acrylic acid salts.

In certain embodiments derivatizing comprises at least one of oxidation and ammoxidation, wherein the oxidation is preferably carried out by gas phase oxidation.

In certain embodiments the bio-propylene oxide is hydrolysed into propylene glycol.

In certain embodiments the monomer blend further comprises further (co)monomer(s) and/or additive(s).

In certain embodiments the further (co)monomers and/or additives are of fossil origin.

In certain embodiments the (co)polymerizing is carried out in the presence of a polymerisation catalyst and/or is initiated by means of a polymerization initiator.

In certain embodiments the (co)polymer composition is polymer composition comprising a homopolymer constituted of bio-propylene units or bio-propylene derivative units, such as a polypropylene, a polyacrylic acid, a polyacrylate, a polyacrolein, a polyacrylonitrile, or a polypropylene glycol, or a copolymer composition comprising a copolymer comprising bio-propylene units and/or bio-propylene derivative units, such as a copolymer comprising bio-acrylic acid and/or bio-acrylate units, a block copolymer comprising bio-propylene oxide units, a polyether polyol, a polyester polyol, an ethylene-propylene-copolymer (EPM), or an ethylene-propylene-diene-copolymer (EPDM). The (co)polymer composition may comprise both a (at least one) homopolymer and a (at least one) copolymer and may comprise further (co)polymer(s) which are not derived by the method of the present invention.

In certain embodiments the monomer blend comprises at least 5 wt.-%, preferably at least 10 wt.-%, at least 20 wt.-%, at least 40 wt.-%, at least 50 wt.-%, at least 60 wt.-%, at least 70 wt.-%, at least 80 wt.-%, or at least 90 wt.-%, even more preferably 100 wt.-% of the bio-monomers, based on the total weight of all monomers in the monomer blend.

In certain embodiments the method further comprises modifying the (co)polymer constituting the (co)polymer composition by side-group hydrolysis and/or derivatisation and/or crosslinking, such intermolecular or intramolecular crosslinking.

In certain embodiments the (co)polymer composition is further processed to produce a sanitary article, a construction material, a packaging material, a coating composition, a paint, a panel, an interior part of a vehicle, such as an interior part of a car, a rubber composition, a tire, a toner, a personal health care article, a part of a consumer good, a part or a housing of an electronic device.

In certain embodiments the method further comprises forming a polymer product, such as a film, beads, a moulded product, a coating composition, a coating, a packaging, a construction material, a rubber composition, a tire, a part of a tire, or a gasket, from the (co)polymer composition optionally together with other components.

The present disclosure further provides a sanitary article, a construction material, a packaging material, a coating composition, a paint, a panel, an interior part of a vehicle, such as an interior part of a car, a rubber composition, a tire, a toner, a personal health care article, a part of a consumer good, a part or a housing of an electronic device, and/or a polymer product obtainable by the method of the present invention.

In certain embodiments the (co)polymer composition is a water-absorbing or rheology-modifying (co)polymer composition comprising acrylic acid.

In certain embodiments the water-absorbing (co)polymer composition is further processed to produce a sanitary article, such as a diaper, a sanitary napkin, an incontinence draw sheet.

In certain embodiments the method further comprises mixing the rheology-modifying (co)polymer composition with further components to produce a coating, a paint, a cosmetic composition.

The present disclosure further provides a (co)polymer composition obtainable by the method of the present invention.

The Bio-Gasoline Component

A bio-gasoline component according to the present disclosure comprises at least 75 wt.-%, preferably at least 85 wt.-%, more preferably at least 90 wt.-% C5-C10 hydrocarbons; at least 8 wt.-%, preferably at least 10 wt.-%, more preferably at least 15 wt.-% cyclic hydrocarbons; n-paraffins, and at least 7 wt.-%, preferably at least 12 wt.-%, more preferably at least 20 wt.-% isoparaffins; and wherein the sum of the wt.-% amounts of isoparaffins and n-paraffins in the bio-gasoline component is at most 65 wt.-%, preferably at most 60 wt.-%, more preferably at most 55 wt.-%; based on the total weight of the bio-gasoline component.

In the context of the present invention, C5-C10 hydrocarbons include any hydrocarbons (molecules consisting of carbon and hydrogen) having at least 5 carbon atoms and at most 10 carbon atoms. Cyclic hydrocarbons in the present invention relate to any hydrocarbons having at least one cycle, including naphthenes and aromatics.

In the bio-gasoline component of the present invention, containing a high amount of iso-paraffins is preferable (thus the relative content of n-paraffins is lowered). In any case, the total content of isoparaffins and n-paraffins should not exceed a certain level, thus improving characteristics of the component. The total paraffins content achievable with the present invention is lower, i.e. more favourable, as compared to a gasoline component obtained by HVO technology predominantly used for manufacturing renewable diesel, by hydrotreating vegetable and/or animal oils, but also providing bio-gasoline components as a by-product.

In certain embodiments the bio-gasoline component has a RON value of at least 60 and a MON value of at least 50, and optionally a RON minus MON value of at least 5. High RON and MON allow blending the bio-gasoline component in higher ratios to gasoline compositions.

In certain embodiments the bio-gasoline component has a 5% boiling point of 50° C. or more and a 95% boiling point of 220° C. or less (ENIS03405).

In certain embodiments the bio-gasoline component comprises at most 1 wt.-% benzene, preferably at most 1 wt.-% (total) aromatics, more preferably at most 0.01 wt.-% (total) aromatics.

In certain embodiments the bio-gasoline component is obtainable by the process of the present disclosure.

In certain embodiments recovering the fraction rich in C5-C10 hydrocarbons as the bio-gasoline component is conducted by distilling the cracking effluent and collecting a fraction having a 5% boiling point of 50° C. or more and a 95% boiling point of 220° C. or less (ENIS03405).

The bio-gasoline component according to the present disclosure can be used in gasoline compositions, or in chemical products intended for industry or households, such as in solvents, thinners and spot removers.

### Examples

The following examples are provided to better illustrate the claimed invention and are not to be interpreted as limiting the scope of the invention. To the extent that specific materials are mentioned, it is merely for purposes of illustration and is not intended to limit the invention.

Preparation and Characteristics of the Hydrotreated Bio-Based Hydrocarbon Feed Samples

Three different hydrotreated bio-based hydrocarbon feed samples (P1, P2 and P3) were prepared by catalytic hydrotreatment, involving hydrodeoxygenation and isomerization reactions, of a fatty feedstock of animal fat and vegetable oils. The hydrotreatment conditions were varied to provide different isoparaffin contents to the hydrotreated bio-based hydrocarbon feed samples. The hydrotreatment effluents were degassed to remove gaseous compounds (NTP), and water vapour, and the liquid effluents were fractionated by distillation collecting distillation cuts having boiling ranges (i.e. initial boiling points (IBP) and final boiling points (FBP)) as reported in table 2. The characteristics of the thus obtained samples P1-P3 were then analysed (tables 1, 3, and 4). Samples P1-P3 were used as catalytic cracking feeds in the inventive catalytic cracking experiments, and samples P2 and P3 as steam cracking feeds in comparative steam cracking experiments. All the hydrotreated bio-based hydrocarbon feed samples had a biogenic carbon content of about 100 wt.-%, based on the total weight of carbon in the hydrotreated bio-based hydrocarbon feed (EN 16640 (2017)).

TABLE 1
Cloud point and density of hydrotreated
hydrocarbon feed samples P1-P3

| Parameter | Method | P1 | P2 | P3 |
|---|---|---|---|---|
| Cloud Point (° C.) | ASTMD7689-17 | 23.1 | −2 | −36 |
| Density (kg/m³) | ENISO12185:1996 | 793.7 | 779.1 | 779.0 |

TABLE 2
Distillation Characteristics (ENISO3405:2019)
of hydrotreated hydrocarbon feed samples P1-P3

| Property | P1 | P2 | P3 |
|---|---|---|---|
| DIS-IBP (° C.) | 273.0 | 194.45 | 177.9 |
| DIS-05 (° C.) | 288.7 | 267.3 | 244.5 |
| DIS-10 (° C.) | 290.6 | 272.5 | 259.4 |
| DIS-20 (° C.) | 292.3 | 277.3 | 269.4 |
| DIS-30 (° C.) | 293.7 | 279.45 | 273.5 |
| DIS-40 (° C.) | 295.2 | 281.45 | 276.2 |
| DIS-50 (° C.) | 296.6 | 283.05 | 278.4 |
| DIS-60 (° C.) | 298.0 | 285 | 280.4 |
| DIS-70 (° C.) | 299.6 | 287.35 | 282.9 |
| DIS-80 (° C.) | 301.5 | 290.6 | 285.9 |
| DIS-90 (° C.) | 303.9 | 293.2 | 289.6 |
| DIS-95 (° C.) | 307.3 | 297.9 | 294.9 |
| DIS-FBP (° C.) | 315.1 | 304.6 | 307.8 |
| DIS-LOSS (vol-%) | 1.1 | 0.8 | 0.5 |
| DIS-RECOVERY (vol-%) | 97.9 | 97.9 | 98.1 |
| DIS-RESIDUE (vol-%) | 1.0 | 1.3 | 1.4 |

Measurement of Isomerization Degree

The compositions of the hydrocarbon feed samples, namely P1, P2, and P3, were analysed by gas chromatography (GC) and were analysed as such, without any pretreatment. The method is suitable for hydrocarbons C2-C36. N-paraffins and groups of isoparaffins (C1-, C2-, C3-substituted and ≥C3-substituted) were identified using mass spectrometry and a mixture of known n-paraffins in the range of C2-C36. The chromatograms were split into three groups of paraffins (C1-, C2-/C3- and ≥C3-substituted isoparaffins/n-paraffin) by integrating the groups into the chromatogram baseline right after n-paraffin peak. N-paraffins were separated from ≥C3-substituted isoparaffins by integrating the n-alkane peak tangentially from valley to valley and compounds or compound groups were quantified by normalisation using relative response factor of 1.0 to all hydrocarbons. The limit of quantitation for individual compounds was 0.01 wt.-%. Settings of the GC are shown in Table 3. The wt.-% amount of n-paraffins and the wt.-% amount of (total) i-paraffins, based on the total weight of the hydrocarbon feed, were determined and are shown in Table 4.

TABLE 3
Settings of GC determination of n- and i-paraffins.

| | GC |
|---|---|
| Injection | split/splitless-injector |
| | Split 80:1 (injection volume 0.2 μL) |
| Column | DB ™-5 (length 30 m, i.d. 0.25 m, |
| | phase thickness 0.25 μm) |
| Carrier gas | He |
| Detector | FID (flame ionization detector) |
| GC program | 30° C. (2 min)-5° C./min-300° C. (30 min), |
| | constant flow 1.1 mL/min) |

TABLE 4
n-paraffin and i-paraffin contents (wt.-%) of
hydrotreated hydrocarbon feed samples P1-P3

| | P1 | | P2 | | P3 | |
|---|---|---|---|---|---|---|
| C. No | nP | iP (total) | nP | iP (total) | nP | iP (total) |
| 2 | 0 | 0 | 0 | 0 | 0 | 0 |
| 3 | 0 | 0 | 0 | 0 | 0 | 0 |
| 4 | 0 | 0 | 0 | 0 | 0.01 | 0 |
| 5 | 0.02 | 0.02 | 0 | 0 | 0.02 | 0.01 |
| 6 | 0.02 | 0.03 | 0.06 | 0.03 | 0.05 | 0.04 |
| 7 | 0.02 | 0.04 | 0.14 | 0.21 | 0.09 | 0.12 |
| 8 | 0.02 | 0.04 | 0.14 | 0.23 | 0.26 | 0.51 |
| 9 | 0.02 | 0.06 | 0.16 | 0.27 | 0.23 | 0.76 |
| 10 | 0.05 | 0.04 | 0.15 | 0.3 | 0.19 | 0.91 |
| 11 | 0.04 | 0.02 | 0.15 | 0.29 | 0.15 | 0.93 |
| 12 | 0.09 | 0.04 | 0.19 | 0.31 | 0.13 | 1.08 |
| 13 | 0.27 | 0.06 | 0.25 | 0.39 | 0.11 | 1.12 |
| 14 | 1.01 | 0.13 | 0.43 | 0.65 | 0.35 | 1.73 |
| 15 | 4.30 | 0.42 | 5.57 | 8.2 | 1.53 | 9.88 |
| 16 | 15.95 | 1.20 | 9.58 | 18.85 | 1.6 | 26.6 |
| 17 | 15.92 | 1.56 | 5.26 | 13.27 | 1.88 | 15.4 |
| 18 | 52.33 | 3.43 | 8.73 | 24.94 | 0.79 | 31.77 |
| 19 | 0.55 | 0.24 | 0.06 | 0.3 | 0.04 | 0.47 |
| 20 | 1.04 | 0.09 | 0.06 | 0.31 | 0.02 | 0.39 |
| 21 | 0.08 | 0.03 | 0.01 | 0.04 | 0.01 | 0.11 |
| 22 | 0.17 | 0.03 | 0.01 | 0.05 | 0.01 | 0.12 |
| 23 | 0.04 | 0.01 | 0.01 | 0.04 | 0.01 | 0.09 |
| 24 | 0.06 | 0.01 | 0.01 | 0.06 | 0.01 | 0.09 |
| 25 | 0 | 0 | 0 | 0 | 0 | 0.01 |
| C25-C29 | 0 | 0.33 | 0 | 0.16 | 0 | 0.32 |
| C30-C36 | 0 | 0.15 | 0 | 0.12 | 0 | 0.07 |
| >C36 | 0 | 0 | 0 | 0 | 0 | 0 |
| Total | 92.00 | 8.00 | 30.96 | 69.04 | 7.48 | 92.52 |

As can be seen from table 4, the hydrotreated bio-based hydrocarbon feed samples P1-P3 were highly paraffinic, and contained about 8 to 93 wt.-% isoparaffins, based on the total weight of the hydrotreated bio-based hydrocarbon feed sample. The hydrocarbon feed samples contained, based on the total weight of the hydrotreated bio-based hydrocarbon feed sample, hydrocarbons having a carbon number of at least C11 as follows: P1 about 100 wt.-%, P2 about 98 wt.-%, and P3 about 97 wt.-%; and C14-C18 hydrocarbons as follows: P1 about 96 wt.-%, P2 about 95 wt.-%, and P3 about 92 wt.-%.

### Comparative Examples—Steam Cracking

Steam cracking experiments were carried out as described in WO 2020/201614 A1, using a bench scale equipment shown in FIG. 1 therein. The main parts of the steam cracking unit, the analytical equipment and the calibration procedure used in these examples have been described in detail in the following publications K. M. Van Geem, S. P. Pyl, M. F. Reyniers, J. Vercammen, J. Beens, G. B. Marin, On-line analysis of complex hydrocarbon mixtures using comprehensive two-dimensional gas chromatography, Journal of Chromatography A. 1217 (2010) 6623-6633 and J. B. Beens, U. A. T. Comprehensive two-dimensional gas chromatography—a powerful and versatile technique. Analyst. 130 (2005) 123-127.

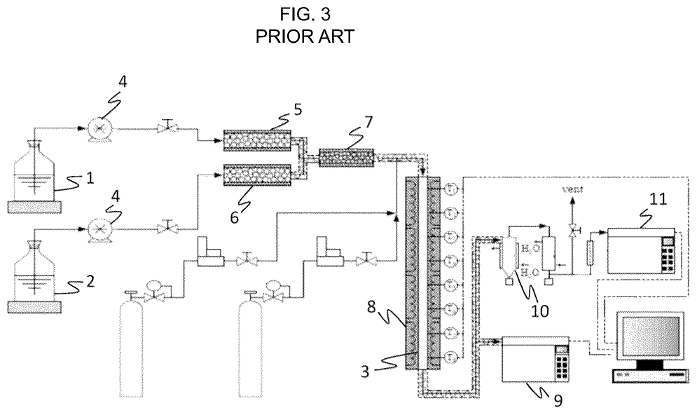

In the following reference is made to the attached FIG. 3 which corresponds to FIG. 1 of WO 2020/201614 A1. The feed section controls the supply of the steam cracking feedstock and the water from reservoirs 1 and 2, respectively, to the reactor coil 3. The flow of liquids was regulated by coriolis flow meter controlled pumps 4 (Bronkhorst, The Netherlands) equipped with Bronkhorst™ CORI-FLOW™ series mass flow metering instruments to provide high accuracy: ±0.2% of reading. CORI-FLOW™ mass flow metering instruments utilizes an advanced Coriolis type mass flow sensor to achieve reliable performance, even with changing operating conditions, e.g. pressure, temperature, density, conductivity and viscosity. The pumping frequency was automatically adjusted by the controller of the CORI-FLOW™ flow metering instrument. The mass flow rate, which contrary to the volume flow rate is not affected by changes in temperature or pressure, of all feeds was measured every second, i.e. substantially continuously. Steam was used as a diluent and was heated to the same temperature as the evaporated feedstock. Both the feedstock and the steam were heated in electrically heated ovens 5 and 6, respectively. Downstream from ovens 5 and 6, the feedstock and the steam were mixed in an electrically heated oven 7 filled with quartz beads, which enabled an efficient and uniform mixing of feedstock and the diluent prior to entering the reactor coil 3. The mixture of feedstock and diluent steam entered the reactor coil 3 placed vertically in a rectangular electrically heated furnace 8. Eight thermocouples T positioned along the axial reactor coordinate measured the process gas temperature at different positions. The rectangular furnace 8 was divided into eight separate sections which could be controlled independently to set a specific temperature profile. The pressure in the reactor coil 3 was controlled by a back pressure regulator (not shown) positioned downstream from the outlet of the reactor coil 3. Two pressure transducers (not shown), placed at the inlet and outlet of the reactor, indicated the coil inlet (CIP) and the coil outlet pressure (COP), respectively. At the reactor outlet, nitrogen was injected to the reactor effluent as an internal standard for analytical measurements and to a certain extent contributes to the quenching of the reactor effluent. The reactor effluent was sampled online, i.e. during operation of the steam cracking setup, at a high temperature (350° C.). Namely, via a valve-based sampling system and uniformly heated transfer lines a gaseous sample of the reactor effluent was injected into a comprehensive two-dimensional gas chromatograph (GC×GC) 9 coupled to a Flame Ionization detector (FID) and a Mass Spectrometer (MS). A high temperature 6-port 2-way sampling valve of the valve-based sampling system was placed in an oven, where the temperature was kept above the dew point of the effluent sample. Further downstream the reactor effluent was cooled to approximately 80° C. Water and condensed heavier products (pyrolysis gasoline (PyGas) and pyrolysis fuel oil (PFO)) were removed by means of a knock-out vessel and a cyclone 10, while the remainder of the effluent stream was sent directly to a vent. Before reaching the vent, a fraction of the effluent was withdrawn for analysis on a Refinery Gas Analyser (RGA) 11. After removal of all remaining water using a water-cooled heat exchanger and dehydrator, this effluent fraction was injected automatically onto the so-called Refinery Gas Analyser (RGA) 11 using a built-in gas sampling valve system (80° C.). The yields of the steam cracking products are reported in table 5.

TABLE 5
Yields of the steam cracking products. The yields are expressed in
wt.-% based on the total weight of the steam cracking effluent.

| Steam cracking feed | P2 | P2 | P2 | P3 | P3 | P3 |
|---|---|---|---|---|---|---|
| Sulphur (ppm) | 250 | 250 | 250 | 250 | 250 | 250 |
| COT (° C.) | 800 | 820 | 840 | 800 | 820 | 840 |
| Dilution (gH₂O/gHC) | 0.5 | 0.5 | 0.5 | 0.5 | 0.5 | 0.5 |
| CO | 0.02 | 0.05 | 0.07 | 0.03 | 0.05 | 0.06 |
| CO₂ | 0.01 | 0.01 | 0.01 | 0.01 | 0.01 | 0.02 |
| C₂H₂ | 0.19 | 0.70 | 0.57 | 0.39 | 0.61 | 0.47 |
| H₂ | 0.40 | 0.50 | 0.60 | 0.45 | 0.54 | 0.60 |
| Methane | 7.99 | 9.75 | 11.00 | 9.38 | 10.80 | 11.74 |
| Ethene | 28.22 | 32.75 | 34.34 | 27.65 | 29.56 | 30.23 |
| Propene | 17.01 | 18.10 | 17.19 | 19.22 | 18.67 | 17.30 |
| 1,3-butadiene | 5.73 | 6.79 | 6.77 | 6.47 | 6.68 | 6.51 |
| non-aromatic C5-C9 | 8.89 | 10.26 | 9.94 | 12.53 | 9.79 | 9.78 |
| Benzene | 2.76 | 3.84 | 6.45 | 4.78 | 6.69 | 7.17 |
| Toluene | 0.94 | 1.40 | 2.03 | 1.95 | 2.73 | 2.58 |
| Xylenes | 0.48 | 0.08 | 0.23 | 0.17 | 0.25 | 0.12 |
| others | 27.38 | 15.78 | 10.80 | 16.98 | 13.64 | 13.40 |
| BTX | 4.17 | 5.33 | 8.71 | 6.9 | 9.66 | 9.88 |
| (benzene, toluene, xylenes) | | | | | | |
| Ethene and Propene | 45.23 | 50.84 | 51.54 | 46.87 | 48.23 | 47.53 |
| HVC (ethene, propene, 1,3- | 55.13 | 62.96 | 67.02 | 60.24 | 64.57 | 63.92 |
| butadiene, and BTX) | | | | | | |
| Total Impurities | 0.22 | 0.75 | 0.65 | 0.44 | 0.67 | 0.55 |
| (CO, CO₂, and C₂H₂) | | | | | | |
| Total Sum of All Species | 100 | 100 | 100 | 100 | 100 | 100 |

Conversion normalized yields were calculated for the steam cracking products by dividing the weight of the steam cracking product by the weight of converted steam cracking feed (i.e. other than unconverted steam cracking feed). For simplicity it is assumed that all unconverted feed material is found in the C10+ fraction, i.e. pyrolysis fuel oil fraction is designated as the unconverted feed, yields of which are presented in table 6. The conversion normalized yields of the different steam cracking products (the weight of the steam cracking product/the weight of converted steam cracking feed) are presented in table 7.

TABLE 6
Yield of the pyrolysis fuel oil fraction, expressed in wt.-%
based on the total weight of the steam cracking effluent.

| Feedstock | P2 | P2 | P2 | P3 | P3 | P3 |
|---|---|---|---|---|---|---|
| Sulphur (ppm) | 250 | 250 | 250 | 250 | 250 | 250 |
| COT (° C.) | 800 | 820 | 840 | 800 | 820 | 840 |
| Dilution (gH₂O/gHC) | 0.5 | 0.5 | 0.5 | 0.5 | 0.5 | 0.5 |
| C10+ = Pyrolysis Fuel Oil | 15.66 | 4.38 | 1.23 | 3.20 | 1.17 | 2.85 |

TABLE 7
Conversion normalized yields of the steam cracking
products, and propylene to ethylene ratio.

| Feedstock | P2 | P2 | P2 | P3 | P3 | P3 |
|---|---|---|---|---|---|---|
| COT (° C.) | 800 | 820 | 840 | 800 | 820 | 840 |
| CO | 0.03 | 0.05 | 0.07 | 0.03 | 0.05 | 0.07 |
| CO₂ | 0.01 | 0.01 | 0.01 | 0.01 | 0.01 | 0.02 |
| C₂H₂ | 0.23 | 0.73 | 0.58 | 0.40 | 0.62 | 0.49 |
| H₂ | 0.47 | 0.53 | 0.60 | 0.46 | 0.54 | 0.62 |
| Methane | 9.47 | 10.20 | 11.14 | 9.69 | 10.92 | 12.08 |
| Ethene | 33.46 | 34.25 | 34.78 | 28.57 | 29.91 | 31.11 |
| Propene | 20.17 | 18.93 | 17.40 | 19.85 | 18.89 | 17.81 |
| 1,3-butadiene | 6.79 | 7.10 | 6.85 | 6.69 | 6.76 | 6.70 |
| non-aromatic C5-C9 | 10.54 | 10.73 | 10.06 | 12.94 | 9.90 | 10.07 |
| Benzene | 3.27 | 4.02 | 6.53 | 4.94 | 6.77 | 7.38 |
| Toluene | 1.11 | 1.47 | 2.06 | 2.01 | 2.76 | 2.66 |
| Xylenes | 0.56 | 0.09 | 0.23 | 0.17 | 0.25 | 0.13 |
| others* | 13.89 | 11.92 | 9.68 | 14.23 | 12.61 | 10.87 |
| BTX (benzene, toluene, xylenes) | 4.95 | 5.33 | 8.71 | 6.90 | 9.66 | 9.88 |
| Ethene and Propene | 53.63 | 50.84 | 51.54 | 46.87 | 48.23 | 47.53 |
| HVC (ethene, propene, 1,3- | 65.36 | 62.96 | 67.02 | 60.24 | 64.57 | 63.92 |
| butadiene, and BTX) | | | | | | |
| C₃H₆:C₂H₄ ratio | 0.60 | 0.55 | 0.50 | 0.69 | 0.63 | 0.57 |

*Since C10+ (=PFO) is considered unconverted material it is removed from this ‘others’ category.

As can be seen from the steam cracking results, the conversion normalized propylene yield is quite similar for P2 and P3, about 20 wt.-%, but the weight ratio of propylene to ethylene is far below 1. Additionally relatively high amounts of methane is formed, which is a strong greenhouse gas with a global warming potential 84 times greater than CO₂ in a 20-year time frame.

Inventive Examples—Fluid Catalytic Cracking

Method

The reactions were carried out in Single Receiver, Short Contact Time, Micro Activity Test (SR-SCT-MAT) apparatus commonly used to benchmark FCC catalysts with the settings shown in table 8 without recycling. A commonly used FCC catalyst was used in the tests.

TABLE 8
Settings of the SR-SCT-MAT apparatus

| | | |
|---|---|---|
| Feed Amount | 10 | mL |
| Injection Time | 300 | s |
| Reaction Temperature | 650° | C. |
| Catalyst Amount | 6-9 | g |

| | |
|---|---|
| WHSV | 12.8 g catalytic cracking |
| | feed/g catalyst per hour |
| Catalyst:Oil Ratio | 1 |

Determination of Yields and Conversion Normalized Yields of the Catalytic Cracking Products

The full results including yields of the catalytic cracking products as wt.-% based on the total weight of the feed, formed coke on the catalyst, weight ratios of certain cracking products, as well as certain characteristics of the gasoline fraction are shown in table 9 below. WHSV of 12.8 g catalytic cracking feed/g catalyst per hour and CAT:OIL=1 are basically equivalent. For simplicity it is assumed in the present invention that all unconverted feed material is found in the LCO and HCO fractions, i.e. the sum of the LCO and HCO fractions is designated as the unconverted feed.

TABLE 9
Results of the catalytic cracking.

| | CONSTANT WHSV | | | | | |
|---|---|---|---|---|---|---|
| | (WHSV = 12.8 g | | | | | |
| | catalytic cracking | | | CONSTANT | | |
| | feed/g catalyst | | | CAT: OIL | | |
| | per hour) | | | (C:O = 1) | | |
| Component | P1 | P2 | P3 | P1 | P2 | P3 |
| Standard Conversion [wt.-% feed] | 78.6 | 59.7 | 40.9 | 79.9 | 61.3 | 41.4 |
| Hydrogen [wt.-% feed] | 0.12 | 0.1 | 0.09 | 0.12 | 0.1 | 0.09 |
| Methane [wt.-% feed] | 0.63 | 0.48 | 0.58 | 0.61 | 0.48 | 0.57 |
| Ethane [wt.-% feed] | 0.85 | 0.63 | 0.63 | 0.83 | 0.62 | 0.62 |
| Ethene [wt.-% feed] | 7.9 | 5.6 | 4.2 | 8.0 | 5.8 | 4.2 |
| Propane [wt.-% feed] | 5.3 | 3.4 | 1.4 | 5.6 | 3.6 | 1.5 |
| Propylene [wt.-% feed] | 24.4 | 18.5 | 12.8 | 24.6 | 18.9 | 13.0 |
| total C4 [wt.-% feed] | 20.7 | 14.6 | 8.6 | 21.2 | 15.0 | 8.7 |
| Std Gasoline (C5-221° C.) [wt.-% feed] | 18.6 | 16.4 | 12.5 | 18.7 | 16.8 | 12.6 |
| Std LCO (221-338° C.) [wt.-% feed] | 20.9 | 40.1 | 58.8 | 19.6 | 38.5 | 58.2 |
| Standard HCO (>338° C.) [wt.-% feed] | 0.49 | 0.21 | 0.37 | 0.49 | 0.2 | 0.37 |
| TOTAL | 99.89 | 100.02 | 99.97 | 99.75 | 100 | 99.85 |

Specific characteristics:

| | | | | | | |
|---|---|---|---|---|---|---|
| Coke [wt.-% feed] | 0.19 | 0.11 | 0.12 | 0.2 | 0.12 | 0.13 |
| Coke On Catalyst [wt.-%] | 0.21 | 0.12 | 0.13 | 0.2 | 0.12 | 0.13 |
| C2 =/total C2 [%] | 90.3 | 89.9 | 86.9 | 90.6 | 90.3 | 87.3 |
| Dry Gas (H₂ + C1-C2) [wt.-% feed] | 9.5 | 6.8 | 5.5 | 9.6 | 7.0 | 5.5 |
| propylene/total C3 [%] | 82.3 | 84.7 | 90.0 | 81.6 | 84.1 | 89.6 |
| propylene/Dry Gas [—] | 2.6 | 2.7 | 2.3 | 2.6 | 2.7 | 2.4 |
| i-Butane [wt.-% feed] | 1.1 | 0.82 | 0.37 | 1.3 | 0.9 | 0.39 |
| n-Butane [wt.-% feed] | 3.4 | 2.1 | 0.79 | 3.6 | 2.2 | 0.84 |
| i-Butene [wt.-% feed] | 5.7 | 4.2 | 2.6 | 5.8 | 4.3 | 2.7 |
| n-Butene | 10.5 | 7.4 | 4.8 | 10.5 | 7.6 | 4.8 |
| C4-Olefins [wt.-% feed] | 16.2 | 11.6 | 7.4 | 16.3 | 11.9 | 7.5 |
| iC4 Olefin/iC4 [—] | 5.1 | 5.1 | 7.2 | 4.5 | 4.7 | 6.9 |
| iC4/total C4 [%] | 5.5 | 5.7 | 4.3 | 6.1 | 6.0 | 4.5 |
| LPG (C3-C4) [wt.-% feed] | 50.4 | 36.4 | 22.8 | 51.4 | 37.5 | 23.2 |
| LPG Olefinicity [%] | 80.6 | 82.8 | 88.7 | 79.7 | 82.1 | 88.2 |
| LPG Olefins [wt.-% feed] | 40.6 | 30.1 | 20.2 | 40.9 | 30.7 | 20.5 |
| LCO-Share [%] | 97.7 | 99.5 | 99.4 | 97.5 | 99.5 | 99.4 |
| Research Octane Number [—] | 66.9 | 67.9 | 71.1 | 68.5 | 69 | 71.2 |
| Motor Octane Number [—] | 58.5 | 60.1 | 63.7 | 60.0 | 61.0 | 63.9 |
| RON-Barrels [—] | 12.4 | 11.2 | 8.9 | 12.8 | 11.6 | 9.0 |
| MON-Barrels [—] | 10.8 | 9.9 | 8.0 | 11.2 | 10.2 | 8.1 |
| n-Paraffins [wt.-% gasoline] | 44.3 | 36.5 | 22.2 | 43.2 | 35.9 | 22.2 |
| Isoparaffins [wt.-% gasoline] | 7.6 | 17.2 | 30.7 | 8.3 | 17.4 | 30.8 |
| Olefins [wt.-% gasoline] | 38.9 | 32.5 | 24.1 | 38.5 | 32.7 | 24 |
| Naphthenes [wt.-% gasoline] | 1.3 | 1.6 | 2.3 | 1.4 | 1.7 | 2.2 |
| Aromatics [wt.-% gasoline] | 7.8 | 12.1 | 20.7 | 8.6 | 12.4 | 20.7 |

Conversion normalized yields of the catalytic cracking products were then calculated by normalising the particular product yield to the fraction of converted catalytic cracking feed (i.e. other than the sum of LCO and HCO fractions that was designated as the unconverted feed here). Conversion normalized yields provide better comparison basis compared to absolute yields, as the unconverted fraction can be easily recovered from the catalytic cracking effluent and recirculated to the cracking reactor, i.e. it does not get wasted but can be eventually converted into cracking products. The conversion normalized yields of the catalytic cracking products (the weight of the catalytic cracking product/the weight of converted catalytic cracking feed), RON and MON of the standard gasoline product fraction, and propylene to ethylene weight ratio in the cracking effluent are reported in table 10.

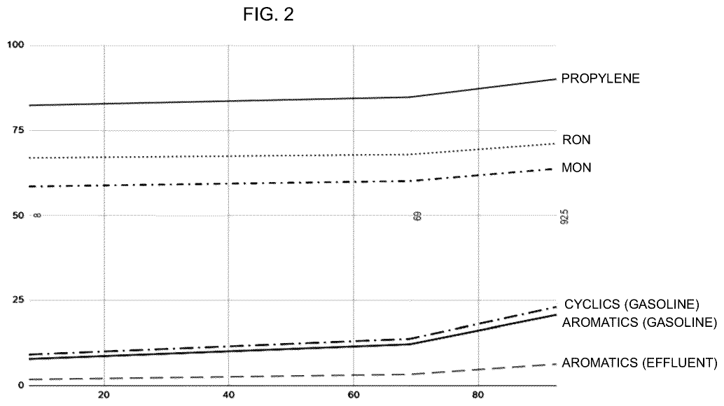

FIG. 2 illustrates selected characteristics of the cracking effluent stream or a specified fraction thereof as a function of isoparaffin content in the catalytic cracking feed (wt.-%). In FIG. 2 Propylene is the weight ratio of propylene to total C3 hydrocarbons (summed amount of propylene and propane) as obtained when using constant WHSV, from Table 9; RON and MON are for the gasoline fraction as obtained when using constant WHSV, from Table 9; Cyclics (gasoline) are the summed amounts of naphthenes and aromatics in the gasoline fraction as obtained when using constant WHSV, from Table 9; Aromatics (gasoline) is the amount of aromatics in the gasoline fraction as obtained when using constant WHSV, from Table 9; and Aromatics (effluent) is the conversion normalized yield of aromatics in the catalytic cracking effluent, from Table 10.

TABLE 10
Conversion normalized yields of the catalytic cracking products,
RON and MON of the standard gasoline product fraction, and
propylene to ethylene ratio in the cracking effluent.

| Component | P1 | P2 | P3 |
|---|---|---|---|
| Hydrogen [wt.-% converted] | 0.2 | 0.2 | 0.2 |
| Methane [wt.-% converted] | 0.8 | 0.8 | 1.4 |
| Ethane [wt.-% converted] | 1.1 | 1.1 | 1.5 |
| Ethene [wt.-% converted] | 10.1 | 9.4 | 10.3 |
| Propane [wt.-% converted] | 6.7 | 5.7 | 3.4 |
| Propylene [wt.-% converted] | 31.0 | 31.0 | 31.3 |
| Aromatics [wt.-% converted] | 1.8 | 3.3 | 6.3 |
| total C4 [wt.-% converted] | 26.3 | 24.5 | 21.0 |
| Standard Gasoline (C5-221° C.) [wt.-% converted] | 23.7 | 27.5 | 30.6 |
| i-Butane [wt.-% converted] | 1.4 | 1.4 | 0.9 |
| n-Butane [wt.-% converted] | 4.3 | 3.5 | 1.9 |
| i-Butene [wt.-% converted] | 7.3 | 7.0 | 6.4 |
| n-Butene [wt.-% converted] | 13.4 | 12.4 | 11.7 |
| Gasoline Research Octane Number [—] | 66.9 | 67.9 | 71.1 |
| Gasoline Motor Octane Number [—] | 58.5 | 60.1 | 63.7 |
| C₃H₆:C₂H₄ [—] | 3.1 | 3.3 | 3.0 |

From the results it can be seen that the inventive catalytic cracking process provides higher conversion normalized yields of propylene (about 31 wt.-%) compared to the comparative steam cracking process (about 20 wt.-%), and that propylene is the main product in the inventive process, while the main product of the comparative process is ethylene. The inventive catalytic cracking process provides also far greater propylene to ethylene ratio (at least about 3) compared to the steam cracking process (well below 1). Furthermore, the inventive catalytic cracking process generates only about 1 wt.-% methane, compared to the about 10 wt.-% of methane generated in the steam cracking process.

While the conversion normalized yields of the propylene remain stable for all the used feeds, surprisingly the conversion normalized yields of aromatics and the bio-gasoline component increase along increasing isomerisation levels in the feed. Also the properties of the bio-gasoline component (RON, MON) improve along increasing isoparaffin content of the feed. Typically in industrial scale processes it is desired to keep the conversion normalized yield of the main product constant. With the present process it is possible to adjust the yields of the further products (gasoline component, aromatics), e.g. depending on market demand of the further products, by simply varying the isoparaffin content of the feed.

The bio-gasoline components obtained in the examples have an elevated content of cyclic hydrocarbons (naphthenes and aromatics), at least 9 wt.-%, and limited content of total paraffins, less than 54 wt.-% with a high share of isoparaffins, compared to typical bio-gasoline components obtainable by conventional HVO technology (technology used for manufacturing renewable diesel by hydrotreating vegetable and/or animal oils, but also providing bio-gasoline components as a by-product). For comparison, a comparative bio-gasoline component was obtained by fractionating a corresponding gasoline fraction from hydrotreated animal fat/vegetable oil, an analysis thereof showing very high paraffin content of 98 wt.-%.

Yet another surprising finding is that, while the conversion normalized yields of propylene remain stable, the conversion normalized yields of propane decrease along increasing isoparaffin content of the feed, meaning that the weight ratio of propylene to the summed amount of propylene and propane in the cracking effluent increases, reaching even 90 wt.-% level. Consequently, high purity propylene composition is obtainable from the catalytic cracking effluent by e.g. simple distillation of C3 hydrocarbons fraction, without necessarily requiring dedicated propylene/propane separation.

1-24. (canceled)

25. A process for manufacturing a bio-propylene composition, and optionally a bio-gasoline component, the process comprising the following steps (B′), (C) and (D):
(B′) providing a catalytic cracking feed including a hydrotreated bio-based hydrocarbon feed containing less than 1 wt.-%, and/or less than 0.8 wt.-%, and/or less than 0.5 wt.-%, of gaseous compounds (NTP);
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in bio-propylene as the bio-propylene composition, and optionally a fraction rich in C5-C10 hydrocarbons as the bio-gasoline component.

26. A process for manufacturing a bio-propylene composition, and optionally a bio-gasoline component, the process comprising the following steps (A) to (D):
(A) hydrotreating an oxygen-containing bio-based feedstock to obtain a hydrotreatment effluent including oxygen-depleted hydrocarbons, and subjecting the hydrotreatment effluent to a gas-liquid separation, and optionally to a fractionation, to provide a hydrotreated bio-based hydrocarbon feed containing less than 1 wt.-%, and/or less than 0.8 wt.-%, and/or less than 0.5 wt.-%, of gaseous compounds (NTP);
(B) providing a catalytic cracking feed including the hydrotreated bio-based hydrocarbon feed;
(C) catalytically cracking the catalytic cracking feed in a catalytic cracking reactor at a temperature of at least 450° C. using a moving solid catalyst to obtain a cracking effluent; and
(D) recovering from the cracking effluent a fraction rich in bio-propylene as the bio-propylene composition, and optionally a fraction rich in C5-C10 hydrocarbons as the bio-gasoline component.

27. The process according to claim 26, wherein the oxygen-containing bio-based feedstock comprises:
one or more selected from the group consisting of vegetable oils, animal fats, microbial oils, thermally liquefied biomass and enzymatically liquefied biomass, preferably one or more selected from the group consisting of vegetable oils, animal fats and microbial oils.

28. The process according to claim 26, wherein the hydrotreating in the step (A) comprises:
at least deoxygenation and isomerization, and/or at least hydrodeoxygenation and isomerization.

29. The process according to claim 25, wherein the hydrotreated bio-based hydrocarbon feed comprises:
iso-paraffins; and the hydrotreated bio-based hydrocarbon feed comprises:
based on a total weight of the hydrotreated bio-based hydrocarbon feed, more than 1 wt.-% isoparaffins, and/or more than 4 wt.-%, and/or more than 5 wt.-% isoparaffins, and/or more than 30 wt.-%, and/or more than 40 wt.-%, and/or more than 50 wt.-%, and/or more than 60 wt.-%, and/or more than 70 wt.-%, and/or 80 wt.-%, and/or more than 85 wt.-% isoparaffins.

30. The process according to claim 25, wherein the hydrotreated bio-based hydrocarbon feed comprises:
isoparaffins and n-paraffins and a sum of the wt.-% amounts of isoparaffins and n-paraffins in the hydrotreated bio-based hydrocarbon feed is at least 40 wt.-%, and/or more than 50 wt.-%, and/or more than 60 wt.-%, and/or more than 70 wt.-%, and/or more than 80 wt.-%, and/or more than 90 wt.-% and/or more than 95 wt.-%, based on a total weight of the hydrotreated bio-based hydrocarbon feed.

31. The process according to claim 25, wherein
the hydrotreated bio-based hydrocarbon feed comprises:
less than 25 wt.-% total aromatics, and/or less than 15 wt.-%, and/or less than 5 wt.-%, and/or less than 1 wt.-% total aromatics, based on a total weight of the hydrotreated bio-based hydrocarbon feed; and/or
the hydrotreated bio-based hydrocarbon feed comprises:
based on a total weight of the hydrotreated bio-based hydrocarbon feed, less than 80 wt.-% naphthenes, and/or less than 50 wt.-%, and/or less than 30 wt.-%, and/or less than 10 wt.-%, and/or less than 5 wt.-%, and/or less than 1 wt.-% naphthenes.

32. The process according to claim 25, wherein the hydrotreated bio-based hydrocarbon feed comprises:
based on the total weight of the hydrotreated bio-based hydrocarbon feed, more than 50 wt.-%, and/or more than 60 wt.-%, and/or more than 70 wt.-%, and/or more than 80 wt.-%, and/or more than 90 wt.-% hydrocarbons having a carbon number of at least 011, and/or at least C14.

33. The process according to claim 25, wherein
(a) the hydrotreated bio-based hydrocarbon feed comprises, based on a total weight of the hydrotreated bio-based hydrocarbon feed:
isoparaffins and n-paraffins, and a sum of wt.-% amounts of the isoparaffins and n-paraffins in the hydrotreated bio-based hydrocarbon feed is at least more than 80 wt.-%, and/or more than 90 wt.-%, and/or more than 95 wt.-%;
more than 80 wt.-%, and/or more than 90 wt.-% hydrocarbons having a carbon number of at least 011, and/or at least C14; and
more than 4 wt.-%, and/or more than 5 wt.-%, and/or more than 30 wt.-% isoparaffins; and/or
(b) the hydrotreated bio-based hydrocarbon feed comprises, based on a total weight of the hydrotreated bio-based hydrocarbon feed:
isoparaffins and n-paraffins, and a sum of wt.-% amounts of isoparaffins and n-paraffins in the hydrotreated bio-based hydrocarbon feed is at least more than 80 wt.-%, and/or more than 90 wt.-% and/or more than 95 wt.-%;
more than 80 wt.-%, and/or more than 90 wt.-%, and/or more than 95 wt.-% hydrocarbons having a carbon number in the range from C5 to 010; and
more than 30 wt.-%, and/or more than 40 wt.-%, and/or more than 50 wt.-% isoparaffins.

34. The process according to claim 25, wherein the hydrotreated bio-based hydrocarbon feed comprises:
based on a total weight of the hydrotreated bio-based hydrocarbon feed, at most 5 wt.-%, and/or at most 3 wt.-%, and/or at most 2 wt.-%, and/or at most 1 wt.-% hydrocarbons having a carbon number of at least C22.

35. The process according to claim 25, wherein the hydrotreated bio-based hydrocarbon feed has a biogenic carbon content, as determined in accordance with EN 16640 (2017), of more than 50 wt.-%, and/or more than 60 wt.-%, and/or more than 70 wt.-%, and/or more than 80 wt.-%, and/or more than 90 wt.-% and/or more than 95 wt.-%, and/or about 100 wt.-%, based on a total weight of carbon in the hydrotreated bio-based hydrocarbon feed.

36. The process according to claim 25, wherein the catalytic cracking feed comprises:
a cracking effluent recycle feed.

37. The process according to claim 36, wherein:
(a) a wt.-% amount of the cracking effluent recycle feed in the catalytic cracking feed is at least 10 wt.-%, and/or more than 20 wt.-%, and/or more than 30 wt.-%, and/or more than 40 wt.-%, and/or more than 50 wt.-%, and/or more than 60 wt.-%, and/or more than 70 wt.-%, and/or more than 80 wt.-%, and/or more than 90 wt.-%, and less than 99 wt.-%, or less than 90 wt.-%, and/or less than 80 wt.-%, and/or less than 70 wt.-%, and/or less than 60 wt.-%, and/or less than 50 wt.-%, and/or less than 40 wt.-%, and/or less than 30 wt.-%, and/or less than 20 wt.-%, based on a total weight of the catalytic cracking feed, and/or from 10 wt.-% to 80 wt.-%; and/or
(b) wherein a sum of the wt.-% amounts of the hydrotreated bio-based hydrocarbon feed and the cracking effluent recycle feed in the catalytic cracking feed is more than 80 wt.-%, and/or more than 85 wt.-%, and/or more than 90 wt.-%, and/or more than 95 wt.-%, and/or more than 97 wt.-%, and/or at least 99 wt.-%, based on a total weight of the catalytic cracking feed; and/or
(c) wherein a weight ratio of and/or hydrotreated bio-based hydrocarbon feed to the cracking effluent recycle feed (hydrotreated bio-based hydrocarbon feed: cracking effluent recycle feed) in the catalytic cracking feed is at least 10:90, and/or at least 20:80, and/or at least 50:50, and/or at least 80:20; and/or at most 99:1, and/or at most 90:10, and/or at most 80:20, and/or at most and/or at most 20:80.

38. The process according to claim 25, wherein the process comprises:
recovering from the cracking effluent a fraction of hydrocarbons having a carbon number of at least C5; and
recycling at least a portion of said fraction to the catalytic cracking feed as a cracking effluent recycle feed.

39. The process according to claim 36, wherein the cracking effluent recycle feed comprises:
based on a total weight of the cracking effluent recycle feed, more than 50 wt.-%, and/or more than 60 wt.-%, and/or more than 70 wt.-%, and/or more than 80 wt and/or more than 90 wt.-% hydrocarbons having a carbon number of at least C5, and/or at least C11, and/or at least C14.

40. The process according to claim 25, wherein a wt.-% amount of the hydrotreated bio-based hydrocarbon feed in a catalytic cracking fresh feed (cracking feed other than optional cracking effluent recycle feed) is more than 80 wt.-%, and/or more than 90 wt.-%, and/or more than 95 wt.-%, and/or at least 99 wt.-%, based on a total weight of the catalytic cracking fresh feed.

41. The process according to claim 25, comprising:
recovering from the cracking effluent a fraction rich in aromatics as a bio-aromatics component.

42. The process according to claim 25, comprising:
recovering from the cracking effluent a fraction rich in bio-ethylene as a bio-ethylene composition and/or including more than 50 wt.-% of ethylene, based on a total weight of the bio-ethylene composition, and/or a fraction rich in C4 hydrocarbons as a bio-C4 composition, and/or including more than 50 wt.-% of C4 hydrocarbons, based on a total weight of the bio-C4 composition, and/or including a fraction rich in C4 olefins as a bio-butylene composition, and/or including more than 50 wt.-% of C4 olefins, based on a total weight of the bio-butylene composition.

43. The process according to claim 25, wherein a recovering, in step (D), comprises:
at least one or more of distilling, fractionating, separating, evaporating, flash-separating, membrane separating, extracting, using extractive-distillation, using chromatography, using molecular sieve adsorbents, using thermal diffusion, complex forming, crystallizing, preferably at least one or more of fractionating, distilling, extracting, and/or using extractive-distillation, and/or at least fractionating.

44. The process according to claim 25, wherein a weight ratio of propylene to ethylene in the cracking effluent is more than 1.0, and/or at least 1.5, and/or more than 2.0, and/or more than 2.5, and/or more than 3.0.

45. 21. The process according to claim 25, wherein the weight ratio of propylene to total-C3 (100%×propylene/{summed amount of propylene and propane}) in the cracking effluent is at least 65 wt.-%, and/or at least 70 wt.-%, and/or at least 80 wt.-%, and/or at least 85 wt.-%, and/or at least 90 wt.-%.

46. A bio-propylene composition comprising:
bio-propylene and bio-propane, wherein a total content of the bio-propylene is at least 80 wt.-%, based on a total weight of the bio-propylene composition, and a weight ratio of bio-propylene to bio-propane is at least 4.5, and/or a total content of the bio-propylene is at least 85 wt.-%, based on a total weight of the bio-propylene composition, and a weight ratio of bio-propylene to bio-propane is at least 5.3, and/or, wherein a total content of the bio-propylene is at least 90 wt.-%, and/or at least 99 wt.-%, based on a total weight of the bio-propylene composition, and a weight ratio of bio-propylene to bio-propane is at least 9.0.

47. A method for producing a (co)polymer composition according to claim 25, the method comprising:
producing a bio-propylene composition; and
optionally purifying the bio-propylene composition, and/or optionally derivatising at least a part of the bio-propylene molecules in the optionally purified bio-propylene composition to obtain a polymerizable composition of bio-monomer(s), and (co)polymerizing a monomer composition including the polymerizable composition of bio-monomers to obtain the (co)polymer composition.

48. The method according to claim 47, wherein:
(a) the polymerizable composition of bio-monomer(s) comprises and/or consists of olefinically unsaturated bio-monomers or epoxide bio-monomers; and/or
(b) the polymerizable composition of bio-monomer(s) comprises and/or consists of at least one olefinically unsaturated bio-monomer selected from a group consisting of bio-propylene, bio-acrylic acid, bio-acrylonitrile, and bio-acrolein, and at least one epoxide bio-monomer selected from a group consisting of bio-propylene oxide; and/or
(c) the monomer composition includes:
other (co)monomer(s) and/or additive(s).

49. The method according to claim 47, wherein:
(a) a weight ratio of propylene to ethylene in the cracking effluent is more than 1.0, and/or at least 1.5, and/or more than 2.0, and/or more than 2.5, and/or more than 3.0; and/or
(b) a weight ratio of propylene to total-C3 (100%×propylene/{summed amount of propylene and propane}) in the cracking effluent is at least 65 wt.-%, and/or at least 70 wt.-%, and/or at least 80 wt.-%, and/or at least 85 wt.-%, and/or at least 90 wt.-%.

50. A bio-gasoline component comprising:
at least 75 wt.-%, and/or at least 85 wt.-%, and/or at least 90 wt.-% C5-C10 hydrocarbons; and/or at least 8 wt.-%, and/or at least 10 wt.-%, and/or at least 15 wt.-% cyclic hydrocarbons; n-paraffins, and at least 7 wt.-%, and/or at least 12 wt.-%, and/or at least 20 wt.-% isoparaffins; and wherein a sum of a wt.-% amounts of isoparaffins and n-paraffins in the bio-gasoline component is at most 65 wt.-%, and/or at most 60 wt.-%, and/or at most 55 wt.-%, based on a total weight of the bio-gasoline component.

51. The bio-gasoline component according to claim 50, wherein
(a) the bio-gasoline component is produced to include:
(b) a bio-gasoline component with a RON value of at least 60; and/or
(c) a bio-gasoline component with a MON value of at least 50; and/or
(d) a bio-gasoline component with a RON minus MON value of at least 5; and/or
(e) a bio-gasoline component with a 5% boiling point of 50° C. or more, and a 95% boiling point of 220° C. or less, as determined in accordance with ENIS03405; and/or
(f) a bio-gasoline component including at most 1 wt.-% benzene, and/or at most 1 wt.-% total aromatics and/or at most 0.01 wt.-% total aromatics.

52. A bio-propylene composition obtained by the process according to claim 25.
